(19) United States
(12) Patent Application Publication
(10) Pub. No.: US 2004/0028733 A1
(43) Pub. Date: Feb. 12, 2004

(54) Polymer-based compositions for sustained release

(75) Inventors: Mark A. Tracy, Arlington, MA (US); Henry R. Costantino, Brookline, MA (US); Maria Figueiredo, Somerville, MA (US); Kevin L. Ward, Arlington, MA (US); David S. Scher, Hudson, MA (US)

Correspondence Address: CAROLYN S. ELMORE, ESQ., ELMORE CRAIG, P.C., 209 MAIN STREET, NO. CHELMSFORD, MA, 01863, US

(73) Assignee: Alkermes Controlled Therapeutics, Inc., Cambridge, MA

(21) Appl. No.: 10/361,797

(22) Filed: Feb. 7, 2003

Related U.S. Application Data
(60) Provisional application No. 60/355,159, filed on Feb. 8, 2002.

(51) Int. Cl. A61K 9/22; A61K 9/14

(52) U.S. Cl. 424/468; 424/486

(57) ABSTRACT

This invention relates to sustained release compositions, and methods of forming and using said compositions, in particular for the sustained release of Follicle Stimulating Hormone (FSH). The sustained release compositions comprise a polymeric matrix of a biodegradable biocompatible polymer and stabilized FSH. The method of the invention for forming a sustained release composition includes, dissolving a biodegradable biocompatible polymer in a polymer solvent to form a polymer solution; adding biologically active stabilized FSH; removing the solvent; and solidifying the polymer to form a polymer matrix containing stabilized FSH dispersed therein. Also described is a method for providing a therapeutically effective amount of stabilized FSH in a patient in need of for a sustained period comprising administering to the patient a dose of the sustained release compositions of the invention. The sustained release composition of FSH can be used to promote maturation of follicles, promote spermatogenesis and to treat fertility disorders.

### RELATED APPLICATION

This application claims the benefit of U.S. Provisional Application No. 60/355,159, filed Feb. 8, 2002. The entire teachings of the above application are incorporated herein by reference.

### BACKGROUND OF THE INVENTION

Follicle Stimulating Hormone (FSH) is a heterodimeric glycoprotein hormone consisting of non-covalently attached α and β subunits. The alpha subunit, a 92 amino acid polypeptide with 5 disulfide bonds, is common to the glycoprotein hormone family, which additionally includes chorionic gonadotropin, thyroid stimulating hormone and luteinizing hormone. The beta subunit, a 111 amino acid polypeptide with 6 disulfide bonds, is unique to FSH. Each subunit has two asparagine-linked glycosylation sites.

Human FSH has been isolated from pituitary glands and from post-menopausal urine (EP 322,438) and has recently been produced recombinantly in mammalian cells (U.S. Pat. Nos. 5,639,640, 5,556,957, 4,923,805, 4,840,896, 5,767,251, EP 212,894 and EP 521,586, see also Howles, C. M., Human Reproduction Update 2(2):172-191 (1996)).

Reproductive function in female and male mammals, including humans, is regulated by FSH. In females, FSH promotes the development of the follicle and consequently ovulation. In males, FSH plays a role in spermatogenesis. The synthesis of FSH by gonadotroph cells takes place within the anterior pituitary gland, before secretion into the general circulation. The synthesis and secretion of FSH are regulated by gonadotrophin releasing hormone, secreted by specialized neurones within the hypothalamus, and steroidal and non-steroidal products secreted from the gonads. Through high-affinity binding to its membrane receptor, FSH affects the function of specific target cells in the ovaries and testes and triggers intracellular mechanisms that regulate steroidogenesis, cell replication, and the expression of specific proteins and growth factors that control gametogenesis.

Difficulties associated with exogenous administration of FSH include a short in vivo half-life requiring frequent, typically daily injections to achieve the desired therapeutic results. Generally such a dosing regime can result in poor patient compliance and consequently unsuccessful treatment. In addition, significant fluctuations of FSH levels in the bloodstream can cause inadequate maturation of the follicles also resulting in unsuccessful treatment.

Therefore, a need exists for FSH sustained release formulations which can lead to greater patient compliance while overcoming the difficulties which can be associated with the administration of FSH.

### SUMMARY OF THE IVENTION

This invention relates to a composition, and methods of forming and using said composition, for the sustained release of FSH. The sustained release composition comprises a biocompatible polymeric matrix of a poly(lactide-co-glycolide) copolymer having a molecular weight from about 5 kD to about 40 kD, preferably, from about 7 kD to about 20 kD, such as from about 10 kD to about 20 kD and having a stabilized FSH formulation, dispersed therein. The stabilized FSH formulation comprises FSH, at least one sugar and optionally at least one buffer salt. The concentration of FSH in the sustained release composition is between about 0.05% (w/w) and about 15% (w/w) of the total weight of the composition. In particular embodiments, the concentration of FSH is between about 0.1% (w/w) and about 1% (w/w). As such, the amount of stabilized FSH formulation needed to achieve this concentration of FSH in the sustained release composition can be determined based on the amount of FSH in the stabilized formulation. The sugar can be a disaccharide, for example, sucrose, lactose or trehalose. The stabilized FSH formulation which is incorporated into the polymer matrix comprises about 30% (w/w) to about 99% (w/w) sugar based on the total dry weight of the stabilized formulation, such as about 50% (w/w) to about 99% (w/w) sugar based on the total dry weight of the stabilized formulation, about 1% to about 70% (w/w) FSH based on the total dry weight of the stabilized formulation, for example, about 1% to about 50% (w/w) FSH, such as about 1% to about 30% FSH, and about 0% to about 25% (w/w) buffer salt based on the total dry weight of the stabilized formulation.

In preferred embodiments, the stabilized FSH formulation which is incorporated comprises 70% to 97% (w/w) sugar, 3% to 30% (w/w) FSH and 0% to 10% (w/w) buffer. The polymer can be a poly(lactide-co-glycolide) copolymer with a terminal methyl ester, a terminal acid group or a blend of the copolymers. The blend can be at a ratio of acid terminal end: ester terminal end for example, 1:3 one acid end to three ester ends. In particular embodiments, the sustained release composition releases FSH in humans over a period of at least five days, preferably the FSH is released for a period of about five days to about thirty days. In preferred embodiments, FSH is released over a period of about five to fourteen days. In other embodiments, the sustained release composition releases FSH in humans for a period of at least 30 days. In preferred embodiments, the composition is in the form of microparticles.

The method of the invention, for forming a composition for the sustained release of FSH includes dissolving a poly(lactide-co-glycolide) copolymer having a molecular weight from about 5 kD to about 40 kD such as from about 7 kD to about 20 kD, for example, from about 10 kD to about 20 kD in a polymer solvent to form a polymer solution, adding the stabilized FSH formulation comprising FSH and at least one sugar to the polymer solution to achieve a polymer/stabilized FSH formulation mixture with a FSH final concentration of between about 0.05% (w/w) and about 15% (w/w) of the dry weight of the composition, removing the polymer solvent thereby forming a solid polymer matrix containing the FSH dispersed therein.

Typically, the stabilized FSH formulation will be added to the polymer solution in the solid form. However, the FSH can be soluble in the polymer solution. That is, the stabilized FSH formulation can be soluble in the polymer solvent or predissolved in an FSH solvent prior to addition to the polymer solution. When an FSH solvent is used and is different from the polymer solvent, all solvents (FSH and polymer) can be removed to form the solid polymer matrix containing the stabilized FSH dispersed therein. When the solvent removed is a combination of the stabilized FSH formulation solvent and polymer solvent, the total solvent is referred to as the solvent phase. When the stabilized FSH formulation is dissolved in a stabilized FSH formulation solvent prior to addition to the polymer solvent the stabilized FSH formulation solvent must be miscible with the polymer solvent, not cause substantial precipitation of the polymer and not be deleterious to the FSH.

Suitable stabilized FSH formulation solvents include, for example: ethanol, methanol, water, acetonitrile, dimethylformamide, dimethylsulfoxide, and combinations thereof.

Suitable solvents for poly (lactide-co-glycolide) include: dimethysulfoxide, ethyl acetate, methylacetate, methylene chloride, chloroform, hexafluoroisopropanol, acetone, and combinations thereof.

The method can further comprise the step of forming droplets of the polymer/stabilized FSH formulation mixture prior to removal of the solvent or solvent phase. Further, the method can comprise freezing the droplets prior to removal of the solvent of solvent phase. According to the method of the invention, the droplets can be microdroplets. In a specific embodiment wherein droplets are formed and then frozen, the polymer solvent or solvent phase can be removed by an extraction process. Alternatively, the polymer solvent or solvent phase can be removed by an evaporation process or a combination of an evaporation and extraction process.

The term “microdroplet” as used herein, refers to a droplet of any morphology which has a dimension less than or equal to about 1,000 microns.

In one embodiment, the method of the invention for using the sustained release composition of FSH, as described herein, comprises administering to a patient in need of treatment a therapeutically effective amount of a composition for the sustained release of FSH, comprising a poly(lactide-co-glycolide) copolymer having a molecular weight from about 5 kD to about 40 kD, preferably, about 7 kD to about 20 kD, such as from about 10 kD to about 20 kD and a stabilized FSH formulation dispersed therein. The stabilized FSH formulation comprises FSH and at least one sugar. The stabilized FSH formulation can optionally include at least one salt, such as a buffer salt. In particular embodiments, the concentration of FSH in the sustained release composition is between about 0.05% (w/w) and 15% (w/w) of the dry weight of the composition. As such, the amount of the stabilized FSH formulation needed to achieve this concentration of FSH in the sustained release composition can be determined based on the amount of FSH in the stabilized formulation.

The amount of FSH present in the stabilized FSH formulation can be from about 1% (w/w) to about 70% (w/w), for example, from about 1% to about 50% (w/w), such as from about 1% to about 30% (w/w). In a particular embodiment, the amount of FSH present in the stabilized FSH formulation can be from about 3% (w/w) to about 30% (w/w) based on the total dry weight of the stabilized formulation.

The concentration of the sugar in the stabilized formulation is about 30% (w/w) to about 99% (w/w) based on the total dry weight of the stabilized formulation, such as from about 50% (w/w) to about 99% (w/w). In particular embodiments, the sugar is present from about 70% (w/w) to about 97% (w/w) based on the total dry weight of the stabilized formulation. In preferred embodiments, the sugar is a disaccharide, such as, lactose, sucrose and trehalose.

The buffer salt is present in the stabilized formulation from about 0% (w/w) to about 25% (w/w) of the total dry weight of the formulation. In a particular embodiment, the buffer salt is present from about 1% (w/w) to about 10% of the total dry weight of the stabilized formulation.

In another embodiment, the invention is a method of promoting or stimulating the maturation of follicles in the ovaries of a patient comprising administering to a patient in need of treatment a therapeutically effective amount of a sustained release composition comprising a poly(lactide-co-glycolide) copolymer having a molecular weight from about 5 kD to about 40 kD, preferably, about 7 kD to about 20 kD, such as from about 10 kD to about 20 kD and a stabilized FSH formulation dispersed therein. The stabilized FSH formulation comprises FSH and at least one sugar. The stabilized FSH formulation can optionally include at least one salt, such as a buffer salt. In particular embodiments, the concentration of FSH in the sustained release composition is between about 0.05% (w/w) and 15% (w/w) of the dry weight of the composition. As such, the amount of the stabilized FSH formulation needed to achieve this concentration of FSH in the sustained release composition can be determined based on the amount of FSH in the stabilized formulation.

The amount of FSH present in the stabilized FSH formulation can be from about 1% (w/w) to about 70% (w/w), for example, from about 1% to about 50% (w/w), such as from about 1% to about 30% (w/w). In a particular embodiment, the amount of FSH present in the stabilized FSH formulation can be from about 3% (w/w) to about 30% (w/w) based on the total dry weight of the stabilized formulation.

The concentration of the sugar in the stabilized formulation is about 30% (w/w) to about 99% (w/w) based on the total dry weight of the stabilized formulation, such as from about 50% (w/w) to about 99% (w/w). In particular embodiments, the sugar is present from about 70% (w/w) to about 97% (w/w) based on the total dry weight of the stabilized formulation. In preferred embodiments, the sugar is a disaccharide, such as, lactose, sucrose and trehalose.

The buffer salt is present in the stabilized formulation from about 0% (w/w) to about 25% (w/w) of the total dry weight of the formulation. In a particular embodiment, the buffer salt is present from about 1% (w/w) to about 10% of the total dry weight of the stabilized formulation.

In yet another embodiment, the invention is a method of promoting spermatogenesis in the testes of a patient comprising administering to a patient in need of treatment a therapeutically effective amount of a sustained release composition comprising a poly(lactide-co-glycolide) copolymer having a molecular weight from about 5 kD to about 40 kD, preferably, about 7 kD to about 20 kD, such as from about 10 kD to about 20 kD and a stabilized FSH formulation dispersed therein. The stabilized FSH formulation comprises FSH and at least one sugar. The stabilized FSH formulation can optionally include at least one salt, such as a buffer salt. In particular embodiments, the concentration of FSH in the sustained release composition is between about 0.05% (w/w) and 15% (w/w) of the dry weight of the composition. As such, the amount of the stabilized FSH formulation needed to achieve this concentration of FSH in the sustained release composition can be determined based on the amount of FSH in the stabilized formulation.

The amount of FSH present in the stabilized FSH formulation can be from about 1% (w/w) to about 70% (w/w), for example, from about 1% to about 50% (w/w), such as from about 1% to about 30% (w/w). In a particular embodiment, the amount of FSH present in the stabilized FSH formulation can be from about 3% (w/w) to about 30% (w/w) based on the total dry weight of the stabilized formulation.

The concentration of the sugar in the stabilized formulation is about 30% (w/w) to about 99% (w/w) based on the total dry weight of the stabilized formulation, such as from about 50% (w/w) to about 99% (w/w). In particular embodiments, the sugar is present from about 70% (w/w) to about 97% (w/w) based on the total dry weight of the stabilized formulation. In preferred embodiments, the sugar is a disaccharide, such as, lactose, sucrose and trehalose.

The buffer salt is present in the stabilized formulation from about 0% (w/w) to about 25% (w/w) of the total dry weight of the formulation. In a particular embodiment, the buffer salt is present from about 1% (w/w) to about 10% of the total dry weight of the stabilized formulation.

In another embodiment, the invention relates to a method of treating fertility disorders. The method comprises administering to a patient in need of treatment a therapeutically effective amount of a sustained release composition comprising a poly(lactide-co-glycolide) copolymer having a molecular weight from about 5 kD to about 40 kD, preferably, about 7 kD to about 20 kD, such as from about 10 kD to about 20 kD and a stabilized FSH formulation dispersed therein. The stabilized FSH formulation comprises FSH and at least one sugar. The stabilized FSH formulation can optionally include at least one salt, such as a buffer salt. In particular embodiments, the concentration of FSH in the sustained release composition is between about 0.05% (w/w) and 15% (w/w) of the dry weight of the composition. As such, the amount of the stabilized FSH formulation needed to achieve this concentration of FSH in the sustained release composition can be determined based on the amount of FSH in the stabilized formulation.

The amount of FSH present in the stabilized FSH formulation can be from about 1% (w/w) to about 70% (w/w), for example, from about 1% to about 50% (w/w), such as from about 1% to about 30% (w/w). In a particular embodiment, the amount of FSH present in the stabilized FSH formulation can be from about 3% (w/w) to about 30% (w/w) based on the total dry weight of the stabilized formulation.

The concentration of the sugar in the stabilized formulation is about 30% (w/w) to about 99% (w/w) based on the total dry weight of the stabilized formulation, such as from about 50% (w/w) to about 99% (w/w). In particular embodiments, the sugar is present from about 70% (w/w) to about 97% (w/w) based on the total dry weight of the stabilized formulation. In preferred embodiments, the sugar is a disaccharide, such as, lactose, sucrose and trehalose.

The buffer salt is present in the stabilized formulation from about 0% (w/w) to about 25% (w/w) of the total dry weight of the formulation. In a particular embodiment, the buffer salt is present from about 1% (w/w) to about 10% of the total dry weight of the stabilized formulation.

### BRIEF DESCRIPTION OF THE DRAWINGS

The foregoing and other objects, features and advantages of the invention will be apparent from the following more particular description of preferred embodiments of the invention, as illustrated in the accompanying drawings

FIG. 1 is a plot of FSH serum concentrations in rats versus time in days following administration of the indicated FSH sustained release compositions.

FIG. 2 is a plot of FSH serum concentrations in rats versus time in days following administration of different doses of the indicated FSH sustained release compositions (dose normalized).

FIG. 3 is a plot of FSH serum concentrations in rats versus time in days following administration of different doses of the indicated FSH sustained release compositions (not dose normalized).

FIG. 4 is a plot of FSH serum concentrations in rats versus time in days following administration of the indicated sustained release compositions.

FIG. 5 is a plot of FSH serum concentrations in rats versus time in days following administration of the indicated sustained release compositions.

FIG. 6 is a plot of FSH serum concentrations in rats versus time in days following administration of the indicated sustained release compositions.

FIG. 7a is a plot of FSH serum concentrations in rats versus time in days following administration of the indicated sustained release compositions.

FIG. 7b is a plot of FSH serum concentrations in humans versus time in days following administration of the indicated FSH sustained release compositions.

### DETAILED DESCRIPTION OF THE INVENTION

A description of preferred embodiments of the invention follows.

The present invention is based on the unexpected discovery that a composition comprising a poly (lactide-co-glycolide) copolymer with a molecular weight from about 5 kD to about 40 kD, preferably, about 7 kD to about 20 kD such as from about 10 kD to about 20 kD and having a stabilized FSH formulation dispersed therein can be used to deliver FSH in a sustained manner. In general, it is desirable that the sustained release of FSH occurs for a period of at least five days.

The sustained release compositions of the invention comprise a biocompatible polymer matrix of a poly(lactide-co-glycolide) copolymer having a molecular weight from about 5 kD to about 40 kD, preferably, about 7 kD to about 20 kD such as from about 110 kD to about 20 kD and having a stabilized FSH formulation dispersed therein. The stabilized FSH formulation comprises FSH and at least one sugar. The sugar can be a disaccharide. The FSH formulation can optionally contain at least one buffer salt. The concentration of FSH in the sustained release composition is between about 0.05% (w/w) and about 15% (w/w) of the total dryweight of the sustained release composition. As such, the amount of stabilized FSH formulation needed to achieve this concentration of FSH in the sustained release composition can be determined based on the amount of FSH in the stabilized formulation.

Typically, the FSH is present in the stabilized formulation from about 1% (w/w) to about 70% (w/w), for example, from about 1% to about 50% (w/w), such as from about 1% to about 30% (w/w). In a particular embodiment, the amount of FSH present in the stabilized FSH formulation can be from about 3% (w/w) to about 30% (w/w) based on the total dry weight of the stabilized formulation. The sugar can be present in the stabilized formulation from about 30% (w/w) to about 99% (w/w) based on the total dry weight of the stabilized formulation, such as from about 50% (w/w) to about 99% (w/w). In particular embodiments, the sugar is present from about 70% (w/w) to about 97% (w/w) based on the total dry weight of the stabilized formulation. The buffer salt can be present in the stabilized FSH formulation from about 0% (w/w) to about 25% (w/w), such as from about 1% (w/w) to about 10% (w/w) of the total dry weight of the stabilized formulation.

The poly(lactide-co-glycolide) (hereinafter “PLG”) can have a lactide:glycolide ratio, for example, of about 10:90, 25:75, 50:50, 75:25 or 90:10. In a preferred embodiment of the invention, the lactide:glycolide ratio of the poly(lactide-co-glycolide) copolymer is 50:50. In certain embodiment, the end groups of the poly (lactide-co-glycolide) are in the methyl ester form. In other embodiments, the end groups of the poly(lactide-co-glycolide) polymer are in the acid form. In further embodiments, the ester form and acid form of the poly(lactide-co-glycolide) can be blended at a suitable ratio. For example, from about 10% of either the ester form or acid form to about 90% of the acid form or ester form, respectively. Preferably, the sustained release composition releases FSH over a period of at least 5 days in humans.

The composition of the present invention as described herein provides a means for eliciting a therapeutic effect in a patient in need thereof by administering a composition comprising poly(lactide-co-glycolide) copolymer and a stabilized FSH formulation dispersed within.

The “stabilized FSH formulation” as defined herein, comprises FSH and at least one sugar. The FSH formulation can optionally contain at least one buffer salt. The stabilized FSH formulation can decrease degradation, aggregation, loss of potency and/or loss of biological activity of the FSH, all of which can occur during formulation of the sustained release composition, and prior to and/or during in vivo release.

A “sugar” as defined herein, is a mono, di or trisaccharide or polyol such as a polysaccharide. Suitable monosaccharides include, but are not limited to glucose, fructose and mannose. A “disaccharide” as defined herein is a compound which upon hydrolysis yields two molecules of a monosaccharide. Suitable disaccharides include, but are not limited to sucrose, lactose and trehalose. Suitable trisaccharides include but are not limited to raffinose and acarbose. It is preferred that the sugar is a non-reducing disaccharide. The amount of sugar present in the stabilized FSH formulation can range from about 30% (w/w) to about 99% (w/w), such as from about 50% (w/w) to about 99% (w/w) of the total dry weight of the stabilized formulation. In particular embodiments, the sugar is present from about 70% (w/w) to about 97% (w/w).

“Buffer salt” as defined herein is the salt remaining following removal of solvent from a buffer. Buffers are solutions containing either a weak acid and a related salt of the acid, or a weak base and a salt of the base. Buffers can maintain a desired pH to stabilize the formulation. For example, the buffer can be monobasic phosphate salt or dibasic phosphate salt or combinations thereof or a volatile buffer such as ammonium bicarbonate. Other buffers include but are not limited to acetate, citrate, succinate and amino acids such as glycine, arginine and histidine. The buffer can be present in the stabilized formulation from about 0% to about 10% of the total weight of the stabilized formulation. In a preferred embodiment, the buffer salt is a sodium phosphate salt.

“Surfactants” as the term is used herein refers to any substance which can reduce the surface tension between immiscible liquids. Suitable surfactants which can be added to the sustained release composition include polymer surfactants, such as nonionic polymer surfactants, for example, poloxamers, polysorbates, polyethylene glycols (PEGs), polyoxyethylene fatty acid esters, polyvinylpyrrolidone and combinations thereof. Examples of poloxamers suitable for use in the invention include poloxamer 407 sold under the trademark PLURONIC® F127, and poloxamer 188 sold under the trademark PLURONIC® F68, both available from BASF Wyandotte. Examples of polysorbates suitable for use in the invention include polysorbate 20 sold under the trademark TWEEN® 20 and polysorbate 80 sold under the trademark TWEEN® 80. Cationic surfactants, for example, benzalkonium chloride, are also suitable for use in the invention. In addition, bile salts, such as deoxycholate and glycocholate are suitable as surfactants based on their highly effective nature as detergents. The surfactant can be present in the polymer phase or present in the stabilized FSH formulation. The surfactant can act to modify release of the FSH from the polymer matrix, can act to stabilize the FSH or a combination thereof.

In addition, other excipients can be added to the polymer phase to modify the release of the FSH from the sustained release composition. Such excipients include salts, such as sodium chloride.

“Antioxidants” can also be added to the sustained release composition. Suitable antioxidants can include, but are not limited to, methionine, vitamin C, vitamin E and maleic acid. The antioxidant can be present in the stabilized FSH formulation or added in the polymer phase. In a particular embodiment, methionine can be added to reduce the oxidation of the disulfides and methionine residues in FSH.

The method of the present invention comprises administering the FSH sustained release compositions described herein to provide a therapeutic or diagnostic effect in a patient in need of such treatment. In preferred embodiments, the sustained release composition can be administered by injection or implantation.

The therapeutic or diagnostic effect can be, for example, the stimulation of ovarian follicular development. Such effects can be useful in women undergoing assisted reproductive technology, including in vitro fertilization (IVF), embryo transfer (EF) and anovulatory infertile women, such as those with polycystic ovary disease. As such, the sustained release composition of the invention comprising a poly (lactide-co-glycolide) copolymer and a stabilized FSH formulation dispersed therein can be used for the treatment of infertility. For example, the sustained release composition having a biocompatible polymer with a stabilized FSH formulation incorporated therein can be suitable for use as a treatment in women with infertility problems related to follicle maturation. To effect final maturation of the follicle and ovulation in the absence of an endogenous LH surge, human chorionic gonadotropin (hCG) can be given simultaneously, following the administration of the sustained release composition, or co-encapsulated with stabilized FSH in the sustained release composition, when monitoring of the patient indicates that sufficient follicular development has occurred.

In another embodiment, the sustained release composition can be administered to the patient to stimulate spermatogenesis in the testes for the treatment of infertility. The release profile for male infertility can be for longer time periods, such as at least 30 days, preferably, for about 60 or about 90 days. In males, FSH blocks inhibition of spermatogenesis by sertolli cells.

Currently, FSH is administered to stimulate ovarian follicular development by daily injection. Daily injections typically results in poor patient compliance. An example of a current daily injection protocol for female infertility is demonstrated in Santibrink and Fauser (Santibrink, E. and Fauser, B. “Urinary Follicle-Stimulating Hormone for Normogonadotropic Clomiphene-Resistant Anovulatory Infertility: Prospective, Randomized Comparison Between Low Dose Step-Up and Step-Down Dose Regimens,” J. Clin. Endocrinology Metab. 82:3597-3602, 1997). As described herein, the FSH sustained release composition can release FSH in a sustained manner for an advantageous period of time.

Further, the sustained release composition can minimize some of the side effects seen with daily administration of FSH, such as Ovarian Hyperstimulation Syndrome which can occur with or without pulmonary or vascular complications. Additionally, the sustained release composition results in a lower initial burst of the FSH when compared to administering FSH alone.

The invention described herein also relates to pharmaceutical compositions suitable for use in the invention. In one embodiment, the pharmaceutical composition comprises a sustained release composition comprising a biocompatible polymeric matrix of poly(lactide-co-glycolide) copolymer having a molecular weight from about 5 kD to about 40 kD, preferably, about 7 kD to about 20 kD such as from about 10 kD to about 20 kD and a stabilized FSH formulation dispersed therein. The concentration of FSH in the sustained release composition is from about 0.05% (w/w) to about 15% w/w. In certain embodiments, the composition can have a period of sustained release in vivo in humans for at least 5 days such as from about 5 days to about 30 days. In other embodiments, such as for women, FSH is released for up to 14 days. In men, FSH can be released for periods of at least 30 days and up to about 90 days, such as about 60 days.

Sustained release of biologically active FSH formulation is a release of the active FSH formulation from a sustained release composition, which occurs over a period of time which is longer than that time period during which a therapeutically significant amount of the biologically active FSH would be available following direct administration of a solution of the biologically active FSH. The resulting in vivo PK profile of FSH from a sustained release composition is also much more consistent (maintained in a desired therapeutic window) than the profile observed following administration of FSH in solution.

Methods of monitoring pharmacokinetics (PK) can be accomplished using widely available techniques such as IRMA analysis of FSH in biological fluids as is described in the Exemplification.

Follicle Stimulating Hormone (FSH), as defined herein, includes all forms of FSH and can be derived from animal, preferably human sources, or recombinantly produced. FSH as defined herein, refers to a compound having the primary, secondary and/or tertiary molecular structure of native FSH, and which has at least one FSH pharmacodynamic effect as measured in standard FSH bioassays. FSH includes analogs, deglycosylated forms, unglycosylated forms and modified glycosylated forms. The most preferred form is produced by recombinant DNA techniques. For Example, GONAL-F® (Ares-Serono) is an example of a currently available FSH formulation given by injection.

“Patient” as that term is used herein, refers to the recipient of the treatment. Mammalian and non-mammalian patients are included. In a specific embodiment, the patient is a mammal, such as a human, canine, murine, feline, bovine, ovine, swine or caprine. In a preferred embodiment, the patient is a human. In a most preferred embodiment, the patient is a female human.

The term “sustained release composition”, as defined herein, comprises poly(lactide-co-glycolide) copolymer having a molecular weight of about 5 kD to about 40 kD, preferably, about 7 kD to about 20 kD, such as about 10 kD to about 20 kD and a stabilized FSH formulation dispersed therein. In a preferred embodiment, the amount of FSH present in the sustained release composition is about 0.05% (w/w) to about 15% (w/w) of the total dry weight composition. As such, the amount of stabilized FSH formulation needed to achieve this concentration of FSH in the sustained release composition can be determined based on the amount of FSH in the stabilized formulation. The amount of FSH administered will vary depending upon the desired effect, patient evaluation, the planned release levels, and the time span over which the FSH will be released.

In alternative compositions, the sustained release composition can contain greater than 15% (w/w) FSH (total dry weight of composition). For example, the alternative compositions can contain 20% to 50% FSH based on the total dry weight of the sustained release composition.

The sustained release compositions of this invention can be formed into many shapes such as a film, a pellet, a rod, a filament, a cylinder, a disc, a wafer, a gel, or a microparticle. A microparticle is preferred. A “microparticle”, as defined herein, comprises a polymer component as described having a diameter of less than about one millimeter and having a stabilized FSH formulation dispersed therein. A microparticle can have a spherical, non-spherical or irregular shape. Typically, the microparticle will be of a size suitable for injection. A preferred size range for microparticles is from about one to about 180 microns in diameter.

The composition of this invention can be administered in vivo, for example, to a human, or to an animal, orally, or parenterally such as by injection, implantation (e.g., subcutaneously, intramuscularly, intraperitoneally, intracranially, and intradermally), administration to mucosal membranes (e.g., intranasally, intravaginally, intrapulmonary, buccally or by means of a suppository), or in situ delivery (e.g., by enema or aerosol spray) to provide the desired dosage of FSH based on the known parameters for treatment with FSH of particular medical conditions.

In preferred embodiments, the sustained release composition, as described herein, is administered by injection. “Injection” as that term is used herein, includes administration through a delivery port alone or in combination with a surgical scope such as a laparoscope, endoscope, laryngoscope, cystoscope, protoscope or thoracoscope. The delivery port can be, for example, a surgical tube such as a catheter with an appropriately sized bore, or a needle or needle-like port. As such, delivery can include a minor incision in the patient to permit entry of a delivery port, such as a needle or catheter, or a combination of a delivery port and a surgical scope. Advantageously, injection of the composition avoids the need for an open surgical procedure to expose the delivery site.

The sustained release of FSH is release of FSH from a polymer matrix comprising a poly(lactide-co-glycolide) polymer having a molecular weight from about 5 kD to about 40 kD, preferably, 7 kD to about 20 kD such as from about 10 kD to about 20 kD which occurs over a period which is longer than that period during which a biologically significant amount of FSH would be available following direct administration of a solution of FSH. In certain embodiments of the present invention, the period of sustained release of FSH occurs over a period of at least 5 days, such as from about 5 days to about 30 days. In other embodiments, the period of sustained release is from about 7 days to about 14 days. In further embodiments, the period of sustained release can be greater than about 30 days such as about 60 days or about 90 days. A sustained release of biologically active FSH, from a sustained release composition can be a continuous or a discontinuous release, with relatively constant or varying rates of release. The continuity of release and level of release can be affected by the polymer composition used (e.g., molecular weight and polymer chemistry, such as choice of end groups and the addition of other excipients which modify release such as surfactants and salts) and FSH loading.

A polymer is biocompatible if the polymer and any degradation products of the polymer are non-toxic to the recipient and also possess no significant deleterious or untoward effects on the recipient's body, such as an immunological reaction at the injection site.

“Biodegradable”, as defined herein, means the composition will degrade or erode in vivo to form smaller chemical species. Degradation can result, for example, by enzymatic, chemical and physical processes.

Suitable biocompatible, biodegradable polymers for alternative embodiments include, for example, poly(lactide), poly(glycolide), poly(lactide-co-glycolide) at molecular weights less than 5 kD or greater than 40 kD, poly(lactic acid)s, poly(glycolic acid), polycarbonates, polyesteramides, polyanhydrides, poly(amino acids), polyorthoesters, poly(dioxanone)s, poly(alkylene alkylate)s, polyetheresters, polyphosphoesters, biodegradable polyurethane, blends thereof, and copolymers thereof.

In further alternative embodiments, suitable biocompatible, non-biodegradable polymers include non-biodegradable polymers selected from the group consisting of polyacrylates, polymers of ethylene-vinyl acetates and other acyl substituted cellulose acetates, non-degradable polyurethanes, polystyrenes, polyvinylchloride, polyvinyl fluoride, poly(vinyl imidazole), chlorosulphonate polyolefins, polyethylene oxide, poly ethylene glycol, poloxamers, polypropylene oxide, blends thereof, and copolymers thereof.

In alternative embodiments, acceptable molecular weights for polymers can be determined by a person of ordinary skill in the art taking into consideration factors such as the desired polymer degradation rate, physical properties such as mechanical strength, and rate of dissolution of polymer in solvent. Typically, an acceptable range of molecular weight is less than 5,000 Daltons or between about 40,000 Daltons to about 2,000,000 Daltons.

The stabilized FSH formulation can be prepared by methods known in the art such as freeze drying, spray-freeze drying, spray drying and those described in U.S. Pat. No. 6,284,283 by Costantino et al. incorporated by reference in its entirety. For example, the stabilized FSH formulation can be prepared by atomizing, using multifluid atomization, a fluid comprising the FSH at least one sugar and at least one solvent at a mass flow ratio of about 0.3 or greater to produce droplets, freezing the droplets to produce frozen droplets, removing the solvent from the frozen droplets to produce friable microstructures, forming a dispersion of the friable microstructures in at least one non-solvent for the FSH and fragmenting the dispersed friable microstructures to produce particles of stabilized FSH formulation.

A number of methods are known by which sustained release compositions (polymer/active agent matrices) can be formed. At a single stage of the process, solvent is removed from the microparticles and thereafter the microparticle product is obtained.

Methods for forming a composition for the sustained release of biologically active agent are described in U.S. Pat. No. 5,019,400, issued to Gombotz et al., and issued U.S. Pat. No. 5,922,253, issued to Herbert et al., the teachings of which are incorporated herein by reference in their entirety.

In this method, a mixture comprising a biologically active agent, a biocompatible polymer such as poly (lactide-co-glycolide) and a polymer solvent such as methylene chloride is processed to create droplets, wherein at least a significant portion of the droplets contains polymer, polymer solvent and the active agent. These droplets are then frozen by a suitable means. Examples of means for processing the mixture to form droplets include directing the dispersion through an ultrasonic nozzle, pressure nozzle, Rayleigh jet, or by other known means for creating droplets from a solution.

Means suitable for freezing droplets include directing the droplets into or near a liquified gas, such as liquid argon or liquid nitrogen to form frozen microdroplets which are then separated from the liquid gas. The frozen microdroplets are then exposed to a liquid or solid non-solvent, such as ethanol, hexane, ethanol mixed with hexane, heptane, ethanol mixed with heptane, pentane or oil.

The solvent in the frozen microdroplets is extracted as a solid and/or liquid into the non-solvent to form a polymer/active agent matrix comprising a biocompatible polymer and a biologically active agent. Mixing ethanol with other non-solvents, such as hexane, heptane or pentane, can increase the rate of solvent extraction, above that achieved by ethanol alone, from certain polymers, such as poly(lactide-co-glycolide) polymers.

A wide range of sizes of sustained release compositions can be made by varying the droplet size, for example, by changing the ultrasonic nozzle diameter. If the sustained release composition is in the form of microparticles, and very large microparticles are desired, the microparticles can be extruded, for example, through a syringe directly into the cold liquid. Increasing the viscosity of the polymer solution can also increase microparticle size. The size of the microparticles which can be produced by this process ranges, for example, from greater than about 1000 to about 1 micrometers in diameter.

When sterile product is desired, the environment in which the process steps are performed can be aseptic. Generally, terminal sterilization of the protein powder or polymer is not recommended. However, the use of Barrier (or Isolator) Technology can provide an aseptic environment. For example, Barrier Technology (Work Station Isolator, LaCalhene, Inc.) can be used to provide an aseptic environment for the manipulation, production and harvesting processes as follows:

1) The Isolator's internal environment (including the surface of any equipment or material packages present) can undergo a decontamination procedure, using vaporized hydrogen peroxide (VHP), a strong oxidizing agent.

2) The decontamination treatment, along with the unit's air filtration system, can ensure the Isolator's internal environment meets and/or exceeds class 100.

3) The operators can be completely segregated from the internal isolator environment through the use of Half Suits and/or glove extensions of the isolator(s).

4) Product contact materials can be sterilized prior to their entry into the isolator via filtration, steam, or dry heat.

5) Materials that are not sterilized during isolator decontamination (See 1, above) can be sterilized prior to their entry into the isolator.

All process gasses and solutions can enter into the isolator via a 0.2 μm sterile filters.

Materials and equipment can be first sterilized in a dry heat oven or autoclave.

If the aforementioned material or equipment is not sterilized as indicated above, the surfaces of these items (or packages of items) can be decontaminated using VHP within a transfer isolator (i.e., small isolator for transferring items to the Work Station Isolator).

The Workstation Isolator can be a flexible walled two half-suit Isolator that can be made of PVC and Divetex. It can have an inlet and outlet HEPA filtration, ventilation/recirculation system and multiple self-propelled fans, which can provide the chamber's turbulent airflow pattern. There can be one transfer port that connects to a VHP-sanitizable freeze dryer. The Isolator can use vapor hydrogen peroxide, which can be generated from a VHP Generator such as those available from Amsco, Inc. using preprogrammed validated cycles, to decontaminate the internal environment. All product manipulation, product transfers and harvest procedures can be performed within the confines of the Isolator.

Yet another method of forming a sustained release composition, from a suspension comprising a poly(lactide-co-glycolide) copolymer and a stabilized FSH formulation, includes film casting, such as in a mold, to form a film or a shape. For instance, after putting the suspension into a mold, the polymer solvent is then removed by means known in the art, preferably at a lowered temperature until a film or shape, with a consistent dry weight, is obtained.

A further example of a conventional microencapsulation process and microparticles produced thereby is disclosed in U.S. Pat. No. 3,737,337, incorporated by reference herein in its entirety, wherein a solution of a wall or shell forming polymeric material in a solvent is prepared. The solvent is only partially miscible in water. A solid or core material is dissolved or dispersed in the polymer-containing mixture and, thereafter, the core material-containing mixture is dispersed in an aqueous liquid that is immiscible in the organic solvent in order to remove solvent from the microparticles.

Another example of a process in which solvent is removed from microparticles containing a substance is disclosed in U.S. Pat. No. 3,523,906, incorporated herein by reference in its entirety. In this process a material to be encapsulated is emulsified in a solution of a polymeric material in a solvent that is immiscible in water and then the emulsion is emulsified in an aqueous solution containing a hydrophilic colloid. Solvent removal from the microparticles is then accomplished by evaporation and the product is obtained.

In still another process as shown in U.S. Pat. No. 3,691,090, incorporated herein by reference in its entirety, organic solvent is evaporated from a dispersion of microparticles in an aqueous medium, preferably under reduced pressure.

Similarly, the disclosure of U.S. Pat. No. 3,891,570, incorporated herein by reference in its entirety, shows a method in which solvent from a dispersion of microparticles in a polyhydric alcohol medium is evaporated from the microparticles by the application of heat or by subjecting the microparticles to reduced pressure.

Another example of a solvent removal process is shown in U.S. Pat. No. 3,960,757, incorporated herein by reference in its entirety.

Tice et al., in U.S. Pat. No. 4,389,330, describe the preparation of microparticles containing an active agent by a method comprising: (a) dissolving or dispersing an active agent in a solvent and dissolving a wall forming material in that solvent; (b) dispersing the solvent containing the active agent and wall forming material in a continuous-phase processing medium; (c) evaporating a portion of the solvent from the dispersion of step (b), thereby forming microparticles containing the active agent in the suspension; and (d) extracting the remainder of the solvent from the microparticles.

Without being bound by a particular theory, it is believed that the release of the FSH can occur by two different mechanisms. First, FSH can be released by diffusion through aqueous filled channels generated in the polymer matrix, such as by the dissolution of the FSH, or by voids created by the removal of the polymer solvent during the preparation of the sustained release composition. A second mechanism is the release of the biologically active agent, due to degradation of the polymer. The rate of degradation can be controlled by changing polymer properties that influence the rate of hydration of the polymer. These properties include, for instance, the ratio of different monomers, such as lactide and glycolide, comprising a polymer; the use of the L-isomer of a monomer instead of a racemic mixture; and the molecular weight of the polymer and the end group chemistry of the polymer (i.e., acid vs. ester). These properties can affect hydrophilicity and crystallinity, which control the rate of hydration of the polymer.

By altering the properties of the polymer, the contributions of diffusion and/or polymer degradation to FSH release can be controlled. For example, increasing the glycolide content of a poly(lactide-co-glycolide) polymer and decreasing the molecular weight of the polymer can enhance the hydrolysis of the polymer and thus, provides an increased biologically active agent release from polymer erosion.

Exemplification

The following methods were employed to analyze samples produced during the production and characterization of the FSH containing sustained release compositions.

Methods of Analysis

Size Exclusion Chromatography (SEC)

Size Exclusion Chromatography was used to quantitate monomeric recombinant human FSH (rhFSH) and to determine the relative amounts of dimeric, monomeric and aggregated rhFSH in samples. A TOSO HAAS column G2000SW×17.8×300, 5μ was utilized. The flow rate was 0.5 mL/min. The mobile phase was 0.1 M Sodium phosphate buffer pH 6.7, 0.1 M sodium sulfate.

Reverse-Phase HPLC (RPHPLC)

Reverse-phase HPLC was used to determine the purity of FSH samples, specifically the amounts of oxidized and native rhFSH. A VYDAC column C4, 4.6 mm, 5 microns, 300 Å was utilized. The eluent system consisted of mobile phase A, 0.1M triethylamine phosphate, mobile phase B, 100% acetonitrile and mobile phase C, 30% acetonitrile and 0.1% TFA employing the following gradient. The flow rate was 1 mL/min.

| | | | % Mobile |
|---|---|---|---|
| (Minutes) | % Mobile Phase A | % Mobile Phase B | Phase C |
| 0 | 86 | 14 | 0 |
| 56 | 72 | 28 | 0 |
| 57 | 0 | 0 | 100 |
| 72 | 0 | 0 | 100 |
| 73 | 86 | 14 | 0 |
| 93 | 86 | 14 | 0 |

SDS-PAGE

SDS-PAGE methods, performed under non-reducing conditions, measured the degree of disassociation of subunits of FSH. Samples were dissolved with sample buffer. The sample was prepared and loaded on Excel gel SDS Gradient 8-18%. The resulting banding pattern was detected by staining with Silver Stain Plus. The purity of the protein was estimated by densitometry and comparison to a standard curve of subunit concentrations. The results were compared to bulk drug carried through the same SDS-PAGE sample preparation procedure.

Particle Size Analysis

The particle size of the microparticles was determined. The mean particle size was determined using a Coulter Multisizer IIe. Briefly, approximately 10 mg of microparticles was added to Isoton® (commercially available buffered saline solution) for particle size analysis. The suspension was then analyzed using a 280 micron aperture tube to determine the particle size distribution. NIST certified polystyrene beads were used as a calibration standard. The results were reported as a volume-weighted median diameter.

Polymer Identity/Gel Permeation Chromatography

Determination of the average molecular weight of the polymer used in the sustained release composition was performed using Gel Permeation Chromatography (GPC). Samples were dissolved in chloroform to approximately 1 mg/mL and resolved on a Polymer Laboratories (Amherst, Mass.) PLgel Mixed-E column (300 mm×7.5 mm, 3 μm particles) using chloroform as the mobile phase. Peaks were detected by a refractive index detector. Polystyrene standards were used.

Load Determination

FSH load in the sustained release compositions was determined by nitrogen analysis using a standard carbon/hydrogen/nitrogen (CHN) elemental analyzer. Samples (approximately 1-5 mg) were combusted at 980° C. in an oxygen atmosphere to produce nitrogen and nitrogen oxides. The gas stream was reduced over copper metal at 700° C. to produce elemental nitrogen that was quantified by a thermal conductivity detector. NIST traceable acetanilide was used as a standard.

MAIACLONE IRMA Assay

The amount of FSH in biological fluids was determined by using a MAIACLONE Kit (BioChem. Immunosystems Italia S.PA.) according to manufacturer's instructions. Briefly, samples, standard and controls are reacted with a mixture of two high affinity monoclonal antibodies; one labeled with radioactive iodine and the other linked to fluorescein. An anti-fluorescein antibody coupled to a magnetic particle is added in excess. This antibody specifically binds to the FSH monoclonal complex and is sedimented in a magnetic field. The concentration of antigen is directly proportional to the radioactivity bound to the magnetic particles. The bound fraction of each sample is measured in a gamma counter calibrated to detect I¹²⁵-Iodine. Using a four parameter fit standard curve, the concentration of FSH in the samples can be determined. The limit of detection is 0.5 mIU/mL and the limit of quantitation is 1.5 mIU/mL for the assay.

Extraction

FSH was extracted from the sustained release compositions utilizing a filter method. The method was used to determine protein integrity and protein content. Briefly, 60 mg of microspheres were suspended in 1 mL of methylene chloride to dissolve the polymer. The suspended protein-containing particles were collected on a 0.65 micron pore size filter. After the filter was allowed to dry, FSH was reconstituted in 1 mL of aqueous media. Following reconstitution, protein integrity and content were determined using SEC, RPHPLC and SDS-PAGE methods already described.

Alternatively, the suspended protein-containing particles were collected by centrifugation, and the supernatant was removed. The pellet was allowed to dry, then reconstituted in 1 mL of aqueous media, and then analyzed for protein integrity and content, as described for the filter method.

Formulation

Formation of a stabilized FSH formulation by lyophilization of a mixture of FSH, sugar and optionally buffers. Freeze drying of the formulation was performed on droplets of the mixture or bulk freeze drying in Lyoguard trays (W. L. Gore and Associates, Delaware). Further details are described herein in Example 5.

When aseptic procedures are desired, the Workstation Isolator and Transfer Isolators, as described above for formation of the microparticles, can be used and can be decontaminated using for example, VHP. The hydrogen peroxide level can be verified as below target level prior to use. The buffers and solutions can be prepared using Water for Injection (WFI). The solution containing FSH and any other desired components can be sterile filtered by, for example, passage through a 0.2 μm filter into the Workatation Isolator. The formulated FSH solution can be sprayed using an atomizing nozzle into a bed of liquid nitrogen. The frozen FSH can then be freeze-dried using a suitable drying cycle. The lyophilized FSH containing powder can then be collected and stored at about −80° C.

Polymers employed were purchased from Alkermes, Inc. of Cincinnati, Ohio.

Polymer 2A (Alkermes, 5050 2A) Poly (lactide-co-glycolide); 50:50 lactide: glycolide ratio; 10 kD Mol. Wt.; carboxylic acid end group.

Polymer 2M (Alkermes, 5050 2M) Poly (lactide-co-glycolide); 50:50 lactide: glycolide ratio; 18 kD Mol. Wt.; methyl ester end group.

Polymer 2A:2M 75% (w/w) 2M and 25% 2A w/w.

Polymer 1A:Alkermes, 5050 1A Poly (lactide-co-glycolide); 50:50 lactide:glycolide ratio, Mol Wt. 5 kD, carboxylic end group

Polymer 1A 45:55 (Alkermes, 45/55 1A) Poly (lactide-co-glycolide); 45:55 lactide:glycolide ratio, Mol Wt. 5 kD, carboxylic end group

Process for Preparing Microparticles

Formation of a polymer solution by dissolving polymer in a suitable polymer solvent.

Addition of the stabilized FSH formulation lyophilizate to the polymer solution to form a polymer/protein mixture.

Optional homogenization of the polymer/protein mixture.

Atomization of the polymer/protein mixture by sonication or other means of droplet formation, and freezing of the droplets by contact with liquid nitrogen.

Extraction of the polymer solvent from the polymer/protein droplets into an extraction solvent (e.g., −80° C. ethanol), thereby forming particles comprising a polymer/stabilized protein matrix.

Isolation of the particles from the extraction solvent by filtration.

Removal of remaining solvent by evaporation.

Sieving of particles by passage through an appropriately sized mesh so as to produce an injectable product.

Aseptic processing:

The Workstation Isolator and Transfer Isolators were decontaminated as described above. The level of hydrogen peroxide level in the isolators was determined. All process liquids were passed through a sterile 0.2 μm filter into the Workstation Isolator. The polymer soltion was prepared by dissolving the polymer in dichloromethan and then filtering it into the Workstation Isolator. The following steps were conducted in the Workstation Isolator: a) The required mass of FSH-containing lyophilized powder was weight out and the required volume of the polymer solution was added to the FSH-containing lyophilized powder. The protein/polymer suspension was sonicated to reduce the particle size of the FSH-containing powder. The protein/polymer suspension was atomized into liquid nitrogen on top of a frozen ethanol bend. The microparticles were cured in the ethanol using a freeze dryer attached to the isolator as a freezer. The slurry of microparticles and ethanol was filtered and the collected microparticles were dried in a sterilized freeze-dryer. The microparticles were sieved inside the Worksation Isolator through a stainless steel sieve.

### EXAMPLE 1

### Effect of PLG Chemistry

The effects of PLG chemistry (end group, molecular weight and lactide-to-glycolide ratio) on recombinant FSH (rhFSH) integrity and release from sustained release compositions were assessed.

TABLE I
PLG Nomenclature

| Formulation | | Lactide: | | Nominal MW |
|---|---|---|---|---|
| Type | Name | glycolide | End Group | (kD) |
| 1 | 2M | 50:50 | methyl ester | 18 |
| 2 | 2A | 50:50 | acid | 10 |
| 3 | 1A | 50:50 | acid | 5 |
| 4 | 45:55 1A | 45:55 | acid | 5 |
| 5 | 2A:2M | 50:50 | acid/ester | 16 |
| | (1:3 blend) | | blend | |

A number of polymers were tested (all at 1.0% rhFSH nominal load), namely, 2M, 2A and 1A, a type 1A polymer with a 45:55 lactide:glycolide ratio and a 2A:2M, 1:3 blend were also investigated.

TABLE II
Effect of PLG type on post-encapsulation
protein integrity and release profile

| | | | For the extracted protein: | | | |
|---|---|---|---|---|---|---|
| | | | Oxidation by | Subunits by | PK Results: | |
| | % rhFSH | PLG | RPHPLC | SDSPAGE | Cₘₐₓ | Duration |
| Formulation | (nitrogen) | Type | (%) | (%) | (mIU/mL) | (day) |
| 1-1 | 1.04 | 2M | 1.4 | 1.2 | 680 ± 70 | up to 36 |
| 2-1 | 1.24 | 2A | 2.0 | 0.94 | 660 ± 30 | 17 |
| 3-1 | 0.96 | 1A | 5.1 | 3.2 | 1000 ± 200 | 14 ± 4 |
| 4-1 | 0.96 | 45:55 | 12.1 | 3.4 | 1300 ± 200 | 10 |
| | | 1A | | | | |

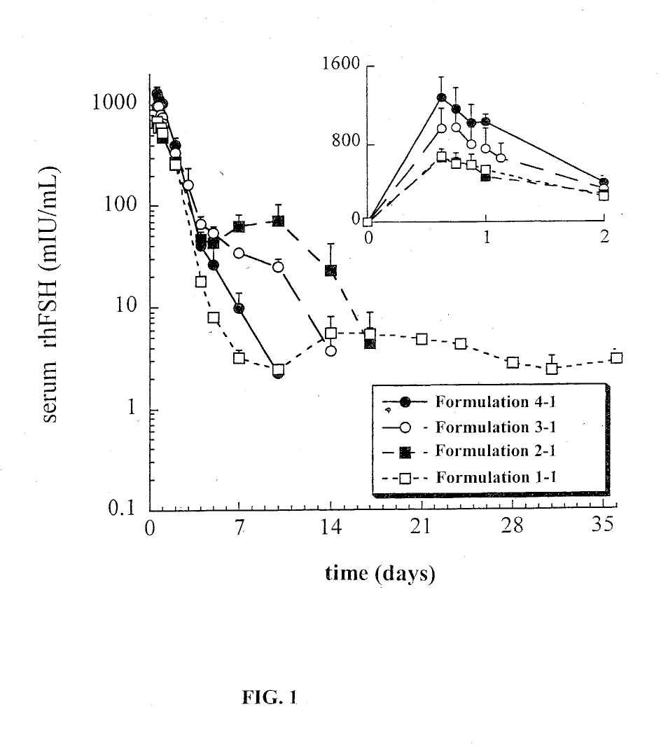

The stabilized FSH formulation was made with 10% FSH, 80% sucrose and 10% phosphate salts. Several different polymer types were screened, as is summarized in Table II. The injected dose was nominally 10 mg of microparticles loaded with 1% rhFSH, or 100 μg protein. PK data are expressed as the data dose-normalized to 200 μg rhFSH/kg rat (FIG. 1). Cₘₐₓ is the experimentally determined as the highest rhFSH concentration that was observed during the study. Duration is defined as the first time point in two consecutive time points with levels below the limit of quantitation, or if there are not two consecutive time points with levels below the limit of quantitation then it is the last time point with a measurable value.

One animal injected with Formulation 1-1 has measurable serum rhFSH levels up to 36 days post-injection (last time point taken) whereas another animal given the same formulation had measurable levels on day 10 and day 31, but not in between, suggesting that in the latter case rhFSH was present in the serum at the end of the study, albeit near the limit of quantitation of the IRMA assay (FIG. 1).

In an additional study, the FSH serum concentration vs. time profile was repeated for Formulation 3-1. This formulation had a Cₘₐₓ of about 825 mIU/mL and a Tₘₐₓ of about 15 hours.

### EXAMPLE 2

### Effect of Excipients

The effect of various excipients co-encapsulated with the lyophilizate during sustained release composition formulation were tested (Table III). Some formulations listed in Table III were made using a sucrose lyophilizate formulation (80:10:10, sucrose:FSH:sodium phosphate salts), as earlier described, and the others were made using a trehalose-containing lyophilizate (trehalose substituted for sucrose).

Various co-encapsulated excipients were tested for their effect on modulating rhFSH release from the sustained release profile of the final composition. Excipients can modulate protein release via various mechanisms, for instance, by enhancing the porosity of the sustained release composition. For example, excipients that have an affinity for water can enhance water sorption into the sustained release composition, and upon dissolution can create additional porosity for protein to be released from the composition. As shown below, the potential release modifiers tested included the salt sodium chloride, and surfactants such as the poloxamer Pluronic F-127, and poly(ethylene glycol), (PEG) 8000 kD MW. These compounds have an affinity for water and cover the range from the highly soluble, low-molecular-weight salt that diffuses rapidly from the microparticles to a soluble polymer (PEG) which should diffuse more slowly out of the microparticles.

The in vivo release profiles of FSH sustained release compositions were analyzed. In a typical PK experiment, three male Sprague-Dawley rats (450±50 g) were injected subcutaneously with 10 mg of microparticles suspended in 0.75 mL of diluent having 3% carboxymethyl cellulose, 0.9% NaCl, and 0.1% TWEEN-20 in water for injection. Serum samples were collected for several time points for the first day, and approximately once per day for up to 35 days, depending on expected duration of the formulation tested. FSH was quantitated from serum samples using the MAIAclone (IRMA) provided by BioChem. Immunosystems Italia S.P.A. Data presented are typically dose-normalized to 200 micrograms FSH/kg.

The rhFSH release profiles demonstrated that the addition of 10% F127, formulation 3-3, or 5% F127 and 5% NaCl, formulation 3-4, to the 1A polymer did not substantially alter the release profile compared to the case of the 1A polymer without the modifier. In all these cases the Cₘₐₓ and duration were similar. At higher concentrations of release modifiers, for example, 10% NaCl in the 1A polymer phase (3-5), and a combination of 5% PEG and 10% NaCl in the 1A polymer (3-7), the duration was reduced by the presence of release modifiers to 14 days and 10 days, respectively. These results can be compared to formulation 3-3, where 10% F127 was added and the sustained release duration was 17 days. A similar result was obtained for release modifiers in the 2A polymer. In the case where 10% NaCl (2-3) or 5% PEG and 10% NaCl, formulation 2-4 were added to 2A polymer microparticles, the last day of measurable rhFSH levels was 17 days post-injection, whereas for formulation 2-2 with 5% F127 and 5% NaCl were added, the duration was 21 days. There were no trends in Cₘₐₓ as a result of the addition of release modifiers. The 1A polymer samples containing release modifiers had a Cₘₐₓ ranging from about 800 to 1100 mIU/mL (Table III), comparable to the release modifier-free sample (Table II). A similar result was seen for the 2A polymer sustained release composition samples.

Table III also presents data for the percent oxidation and percent subunit formation of the protein following extraction from the microparticles using the methylene chloride centrifugation method. The formation of subunits was very low, 1% or less, in all formulations. The SEC data, not shown, demonstrated about 100% monomeric protein for all samples. The oxidation for all formulations was 3-5% for the formulations containing release modifiers.

TABLE III
Effect of release modifiers on post-encapsulation
protein integrity and release profile

| | | | | | For the extracted | | | |
|---|---|---|---|---|---|---|---|---|
| | | | | | protein: | | | |
| | | | Stabilized | Co- | | Sub | PK Analyses: | |
| | % | PLG | FSH | sprayed | Oxidation | units by | C^{max} | Duration |
| Formulation | RhFSH | Type | Formulation | Additive | by RPHDLC % | SDSPAGE | (mIU/mL) | (Day) |
| 2-2 | 1.0 | 2A | 80:10:10 | 5% | 4.9 | 0.8 | 620 ± 70 | 21 |
| | | | sucrose:FSH: | F127/5% | | | | |
| | | | sod. phos. salt | NaCl | | | | |
| 2-3 | 1.1 | 2A | 80:10:10 | 10% | 4.9 | 1.0 | 900 ± 20 | 17 |
| | | | sucrose:FSH: | NaCl | | | | |
| | | | sod. phos. salt | | | | | |
| 3-2 | 1.4 | 2A | 80:10:10 | 5% PEG | 4.5 | 0.4 | 710 ± 100 | 17 |
| | | | trehalose:FSH: | 10% | | | | |
| | | | sod. phos. salt | NaCl | | | | |
| 3-3 | 1.0 | 1A | 80:10:10 | 10% | 3.2 | 0.4 | 1100 ± 50 | 17 |
| | | | trehalose:FSH: | F127 | | | | |
| | | | sod. phos. salt | | | | | |
| 3-3 | 1.2 | 1A | 80:10:10 | 5% | 5.2 | 0.8 | 1100 ± 150 | 17 |
| | | | sucrose:FSH: | F127/5% | | | | |
| | | | sod. phos. salt | NaCl | | | | |
| 3-4 | 1.2 | 1A | 80:10:10 | 10% | 4.5 | 1.3 | 1000 ± 100 | 14 |
| | | | sucrose:FSH: | NaCl | | | | |
| | | | sod. phos. salt | | | | | |
| 3-5 | 1.1 | 1A | 80:10:10 | 5% PEG | 5.0 | 0.5 | 1100 ± 100 | 16 ± 7 |
| | | | trehalose:FSH: | | | | | |
| | | | sod. phos. salt | | | | | |
| 3-6 | 1.4 | 1A | 80:10:10 | 5% PEG | 4.6 | 0.4 | 800 ± 60 | 10 |
| | | | trehalose:FSH: | 10% | | | | |
| | | | sod. phos. salt | NaCl | | | | |

### EXAMPLE 3

### Effects of Protein Load, Dose and Sucrose VS. Trehalose as Stabilizing Excipient

The effect of protein loading on protein integrity and release was assessed. In addition to the load of 1% discussed above, lower FSH loads of 0.5% and 0.25% were also tested. PK studies were conducted for the same 100 μg dose of rhFSH per rat, corresponding to administration of 10, 20 and 40 mg of the sustained release composition, respectively. This study utilized a sucrose-containing lyophilizate formulation for the 2A and 2M polymer types. In addition, the same load-ranging series of polymer types was produced with the alternate trehalose lyophilizate. Data are presented in Table IV.

TABLE IV
Effect of load and stabilizer on post-encapsulation
integrity and release profile

| | | | | For the extracted | | |
|---|---|---|---|---|---|---|
| | % | | | protein: | | PK analyses:^{a} |
| | Load | PLG | Stabilized | Oxidation | Subunits | Cₘₐₓ |
| Formulation | rhFSH | Type | FSH Formulation | by RPHPLC % | by SDSPAGE % | (mIU/mL) |
| 1-1 | 1.04 | 2M | sucrose | 1.4 | 1.2 | 680 ± 70 |
| 1-2 | 0.48 | 2M | sucrose | 1.6 | 1.4 | 400 ± 10 |
| 1-3 | 0.27 | 2M | sucrose | 2.3 | 2.2 | 360 ± 30 |
| 1-4 | 0.92 | 2M | trehalose | 2.3 | 0.7 | 690 ± 10 |
| 1-5 | 0.48 | 2M | trehalose | 1.6 | 0.7 | 220 ± 30 |
| 1-6 | 0.25 | 2M | trehalose | 1.8 | 1.1 | 220 ± 20 |
| 2-1 | 1.24 | 2A | sucrose | 2.0 | 0.9 | 660 ± 30 |
| 2-5 | 0.64 | 2A | sucrose | 2.2 | 1.6 | 320 ± 150 |
| | | | | | | 280 ± 3^{b} |
| 2-6 | 0.32 | 2A | sucrose | 3.2 | 3.6 | 300 ± 30 |
| 2-7 | 0.75 | 2A | trehalose | 4.2 | 1.4 | 900 ± 60 |
| 2-7 | 0.53 | 2A | trehalose | 3.4 | 2.0 | 340 ± 20 |
| | | | | | | 320 ± 20^{b} |
| 2-7 | 0.25 | 2A | trehalose | 4.2 | 2.1 | 290 ± 30 |

^{a}Injected dose was nominally 100 μg protein except as noted.
^{b}Injected dose was nominally 50 μg protein.

All samples in Table IV contained about 100% monomeric protein by SEC and up to about 4% oxidized and subunit species. No trends were observed between the protein stability and load or choice of sugar, for example, the sugar can be a disaccharide such as sucrose or trehalose. The release profiles were similar. Therefore, both sucrose and trehalose are suitable stabilizers for rhFSH upon lyophilization and subsequent encapsulation in PLG sustained release compositions.

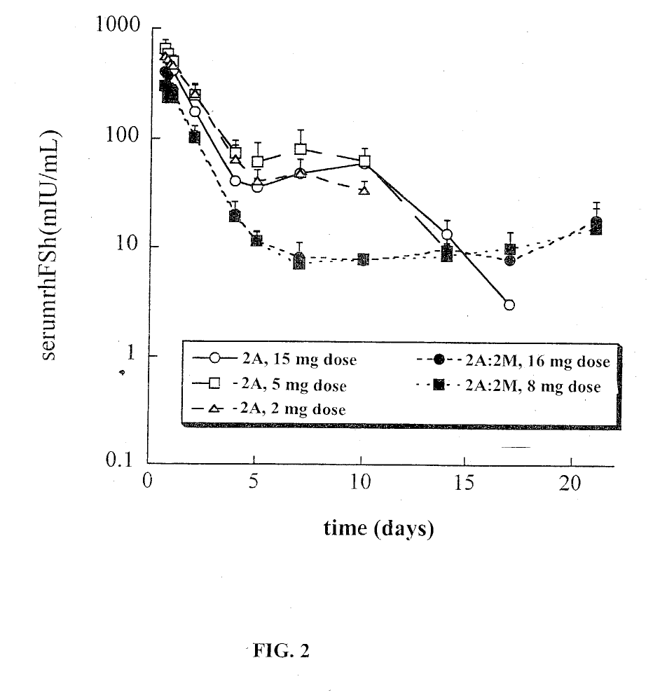

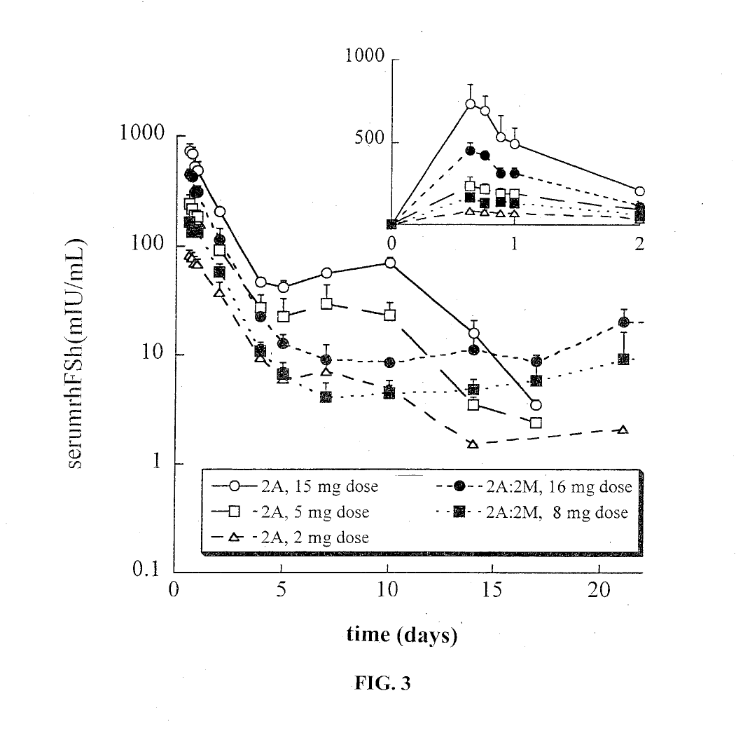

Modification of the dose of encapsulated rhFSH was also studied. For the sucrose-containing lyophilizate loaded at 0.5% rhFSH in 2A polymer, administering sustained release composition doses (nominally) of 15 mg, 5 mg and 2 mg were tested. The serum profiles out to 21 days are shown in FIG. 2. Upon normalization, all doses for the 2A polymer sustained release compositions from 2-15 mg (10-75 μg rhFSH/rat) yielded similar release profiles over 7 days. 16 mg and 8 mg of the 2A:2M polymer blend formulation (Type 5) using the same lyophilizate and load were also administered. Serum data for these doses of the blend are shown in FIG. 2. Upon normalization to the same dose, the rhFSH levels were comparable for the 16 mg and 8 mg doses. In addition, the release profiles for the 2A:2M polymer blend sustained release compositions were distinctly different than the 2A polymer sustained release compositions, regardless of the dose. These same data are also presented in non dose-normalized form in FIG. 3. Data beyond 17 days for all 2A doses were below the assay quantitation limit.

### EXAMPLE 4

### Characterization of rhFSH Sustained Release Composition

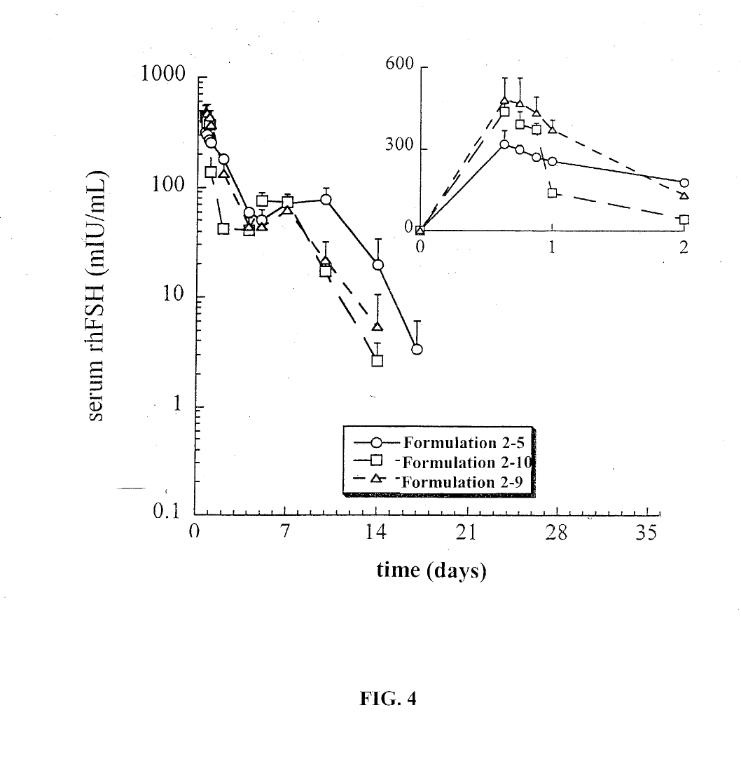

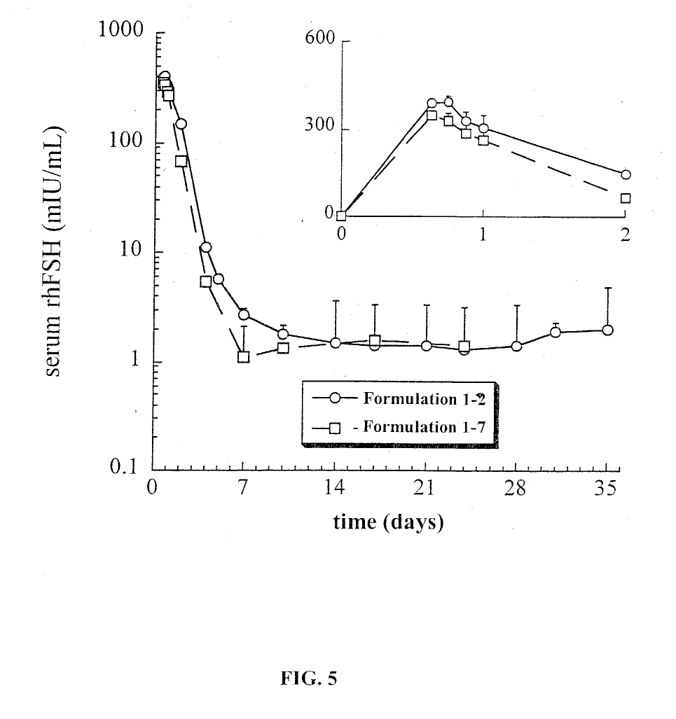

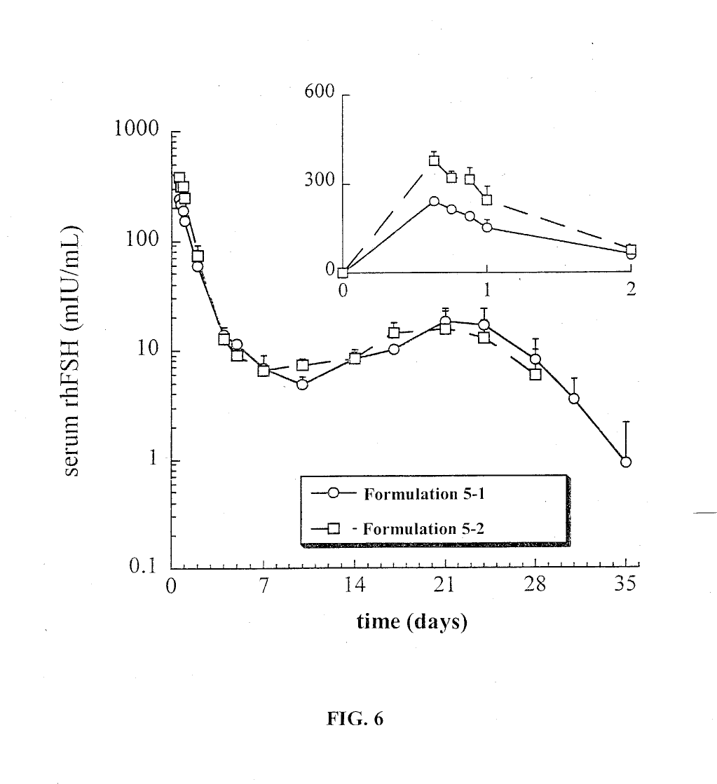

Table V presents data for a number of batches of three formulations of rhFSH sustained release compositions (all at 0.5% protein load, three different polymers, namely, 2M, 2A and a 1:3 (w/w) blend of 2A:2M, formulations 1, 2 and 5 respectively). These sustained release compositions batches were made using the sucrose-containing lyophilizate formulation (80:10:10; sucrose, FSH, phosphate salts). The data for extracted protein were generated using the filter extraction method (except where so noted in Table V). In addition to integrity data for the extracted protein (using the filter method), the Table also presents sustained release composition characterization data: median particle size (D_{v.50}) and moisture content. For comparison, Table V also contains data from Example 1 of 2A (formulation 2-5), 2M (formulation 1-2) and the 2A:2M blend formulation (5-1). The data show that stability of rhFSH towards encapsulation was similar to that observed in earlier batches of the same formulation. For all batches, the median microparticle size was about 40-60 μm. The range of water content for all polymer batches was between about 0.5 and 1.0%. Release profiles for various batches of the lead rhFSH microparticle formulations are shown in FIG. 4 (2A polymer formulation), FIG. 5 (2M polymer formulation) and FIG. 6 (2A:2M polymer blend formulation). FIGS. 4, 5 and 6 and Table V show that the in vitro and in vivo results are similar between the batches of each formulation.

TABLE V
Characterization of microparticles containing a load of 0.5% protein
formulated as 80:10:10 sucrose:protein:sodium phosphate

| | | | | | For the extracted protein: | | |
|---|---|---|---|---|---|---|---|
| | | | | | Monomer | | Subunits by |
| | % | Polymer | D_{v.50} | H₂0 | by SEC | Oxidation | SDSPAGE |
| Formulation | rhFSH | Type | (μm) | (%) | (%) | by RPHPLC (%) | (%) |
| 1-2 | 0.48 | 2M | 58.7 | 0.52 | 100^{a} | 1.6^{a} | 1.4^{a} |
| 1-7 | 0.59 | 2M | 41.7 | 0.57 | 99.5 | 2.4 | <0.25 |
| 1-8 | 0.51 | 2M | 51.7 | 1.54 | na | na | na |
| 1-9 | 0.61 | 2M | na | 0.70 | 99.6 | 3.1 | <0.25 |
| 1-10 | 0.61 | 2M | 51.8 | 0.65 | 99.7 | 2.8 | <0.25 |
| | | | 56.0 | | | | |
| 2-4 | 0.64 | 2A | 67.7 | 0.84 | 100^{a} | 2.2^{a} | 1.6^{a} |
| 2-9 | 0.51 | 2A | 59.5 | 0.73 | 99.7 | 3.1 | <0.25 |
| 2-10 | 0.59 | 2A | 39.9 | 0.90 | 99.7 | 6.6 | <0.25 |
| 2-11 | 0.48 | 2A | 54.0 | 0.84 | 99.7 | 4.3 | <0.25 |
| | | | 46.9 | | | | |
| 2-12 | 0.61 | 2A | na | na | 99.8 | 6.9 | <0.25 |
| 2-13 | 0.48 | 2A | 64.0 | 0.68 | 99.7 | 5.5 | <0.25 |
| 1-10 | 0.61 | 2M | 51.8 | 0.65 | 99.7 | 2.8 | <0.25 |
| | | | 56.0 | | | | |
| 5-1 | 0.61 | 2A:2M | na | 0.37 | 99.7^{a} | 1.2^{a} | 0.8^{a} |
| | | 1:3 | | | | | |
| 5-2 | 0.56 | 2A:2M | 54.2 | 0.85 | 99.6 | 2.6 | <0.25 |
| | | 1:3 | | | | | |
| 5-3 | 0.61 | 2A:2M | na | na | 99.8 | 2.8 | <0.25 |
| | | 1:3 | | | | | |
| 5-4 | 0.59 | 2A:2M | 42.0 | 0.59 | 99.7 | 2.8 | <0.25 |
| | | 1:3 | | | | | |

^{a}Data from a centrifuge-extracted sample
^{b}na = not available

### EXAMPLE 5

### Stabilized FSH Formulation

The stabilized lyophilizate used in the sustained release composition described above were identified by their ability to minimize degradation, aggregation, loss of potency and/or loss of the FSH, all of which can occur during formulation of the sustained release composition. Stable FSH-containing lyophilizates which were subsequently encapsulated in polymer were produced. Lyophilizate formulation studies were conducted to identify additives that stabilize FSH through the spray-freeze drying processing step and to assess the stability of the lyophilizates after being exposed to moisture at a physiologic temperature, a condition mimicking the early stages of protein dissolution and release from microparticles.

Seven FSH-containing lyophilizates were produced to identify salts and additives that stabilized FSH formulation through lyophilization and after exposure to moisture. Each lyophilizate contained 10% FSH, 10% salt, and the remainder (80%) a stabilizing additive. The lyophilizate composition was determined based on an estimated total load of 100 μg FSH to be delivered in one weekly injection and a target lyophilizate load (FSH mass+salt+stabilizer) of 10% in 10 mg of microparticles. The formulations are summarized in Table VI.

TABLE VI
Components of Initial Lyophilizate Formulations

| Formulation | Protein | Salt | Additive |
|---|---|---|---|
| L-1 | FSH | Sodium Phosphate | Sucrose |
| L-2 | FSH | Sodium Bicarbonate | Sucrose |
| L-3 | FSH | Sodium Phosphate | Trehalose |
| L-4 | FSH | Sodium Bicarbonate | Trehalose |
| L-5 | FSH | Sodium Phosphate | Ammonium Sulfate |
| L-6 | FSH | Sodium Bicarbonate | Ammonium Sulfate |
| L-7 | FSH | Sodium Bicarbonate | Zinc Acetate |

The formulations were prepared by adding solutions of the additive and salt to the bulk drug. Each formulated solution was then spray-freeze dried to produce a lyophilized powder. A sample of each powder was dissolved in DI water and evaluated by SEC to assess post-lyophilization stability. In addition, each powder was exposed to 100% relative humidity for 24 hours in 37° C. and subsequently evaluated by SEC to assess post-humidification stability. The results from these experiments are given in Tables VII and VIII, respectively.

TABLE VII
Effect of Formulation Components on
FSH Stability After Lyophilization

| | | | % Recovered | |
|---|---|---|---|---|
| | Nominal | Measured | (Measured/ | |
| Formu- | Concentration | Concentration | Nominal × | |
| lation | (μg/mL) | (SEC) (μg/mL) | 100) | % Monomer |
| L-1 | 150 | 137 | 91 | 100 |
| L-2 | 150 | 146 | 98 | 100 |
| L-3 | 150 | 153 | 102 | 100 |
| L-4 | 150 | 165 | 110 | 100 |
| L-5 | 150 | 176 | 117 | 100 |
| L-6 | 150 | 180 | 120 | 100 |
| L-7 | 150 | 99 | 66 | 100 |
| | | | | peak tailing |

The data in Table VII show that FSH is stable through lyophilization as assessed by SEC for 6 of 7 formulations. The low recovery and peak tailing observed in the chromatogram for sample L-7 (w/zinc acetate) may be indicative of degradation.

TABLE VIII
Effect of Formulation Components on FSH Lyophilizate
Stability After Exposure to Moisture at 37° C.

| | | | % Recovered | |
|---|---|---|---|---|
| | Nominal | Measured | (Measured/ | |
| Formu- | Concentration | Concentration | Nominal × | |
| lation | (μg/mL) | (SEC) (μg/mL) | 100) | % Monomer |
| L-1 | 150 | 127 | 85 | 100 |
| L-2 | 150 | 145 | 97 | 99 |
| L-3 | 150 | 157 | 105 | 100 |
| L-4 | 150 | 134 | 89 | 99 |
| L-5 | 150 | 25 | 16 | 11 |
| L-6 | 150 | 27 | 18 | 11 |
| L-7 | 150 | 92 | 62 | 95 |

Table VIII shows that the salt and additive have significant effects on the stability of FSH after humidification. The data suggest that these formulations are stable through lyophilization and humidification. Both the sucrose and trehalose formulations made with bicarbonate showed a small peak at 13.7 minutes by SEC suggesting the presence of aggregates. Formulations L-5, L-6 and L-7 had aggregates and/or subunits. Ammonium sulfate appears to be a strongly destabilizing additive for FSH.

Based on these results, trehalose/phosphate and sucrose/phosphate lyophilizate formulations have been identified as stable lyophilizates.

The stabilized FSH formulation is prepared from a formulated aqueous solution containing FSH, a stabilizing excipient (e.g., sugar) and possibly at least one buffer salt The formulated aqueous solution can be dried into a friable form suitable for processing to produce sustained release compositions by a variety of pharmaceutical processing methods such as bulk freeze drying, spray drying, spray-freeze drying, rotary evaporation vacuum drying, and supercritical fluid drying. Spray-freeze drying in particular is suitable for production of highly friable dried solids that, according to the processing conditions, can yield micron down to sub-micron powders (Costantino et al., U.S. Pat. No. 6,284,283, incorporated herein by reference). Somewhat less friable powders can be achieved by bulk freeze drying. In a preferred embodiment, the formulated aqueous solution can be poured into a container, for example a LYOGUARD tray (W. L. Gore & Associates, Elkton, Md.), frozen on the lyophilizer shelf, and dried in a lyophilizer. In another preferred embodiment, the formulated aqueous solution in sprayed into a freezing medium (e.g., liquid nitrogen) using an atomization technique (e.g., single fluid, high pressure nozzle) and the liquid nitrogen slurry is poured into the container, and the frozen material dried by lyophilization in a lyophilizer. The latter embodiment allows for production of powders with larger particle size compared to those generated by spray-freeze drying.

Comparison of the particle sizes generated using these different approaches for the 10% FSH, 80% sucrose and 10% phosphate salt stabilized FSH formulation is presented in the following table:

TABLE IX

| Liquid Processing Method | Particle Size (D_{v,50} in microns) |
|---|---|
| Two fluid atomization (1) | 0.41 |
| Two fluid atomization (2) | 0.47 |
| Bulk frozen in LYOGUARD tray (1) | 7.7-10.4 |
| Bulk frozen in LYOGUARD tray (2) | 9.6 |
| Bulk frozen in LYOGUARD tray (3) | 7.3-9.0 |
| Flash frozen (single fluid atomization) | 0.7-2.0 |

### EXAMPLE 6

### Pharmacokinetic Studies of FSH Unencapsulated

The pharmacokinetics (PK) of recombinant human Follicle Stimulating Hormone (rhFSH) (Serono) bulk doses were evaluated in male Sprague-Dawley rats weighing about 400 g±50 g (SD) to demonstrate the ability to detect FSH in serum. For rhFSH PK characterization the doses were:

A) 0.5 μg/0.5 mL as an intravenous (IV) bolus

B) 10 μg/0.5 mL, 5 μg/0.5 mL and 1 μg/0.5 mL as a subcutaneous bolus (SC)

C) 0.5 μg/hr (0.5 μg/μL), 0.25 μg/hr (0.25 μg/μL) and 0.05 μg/hr (0.05 μg/μL) as a continuous SC delivery from an ALZET® osmotic pump (Model 2001, 1.011/hr, 1 week duration). The vehicle for the IV and SC bolus studies, and the SC osmotic pump study was 0.9% saline.

Blood samples were obtained at various time points and serum was separated for assay. Samples were analyzed according to the immunoradiometric assay (IRMA) method. The following parameters for rhFSH were estimated: distribution and elimination phase half-lives (alpha and beta HL), area under the serum concentration-time curve (AUC) from time zero to infinity, maximum observed concentration (Cₘₐₓ) time for the maximum observed concentration (Tₘₐₓ) and average steady-state concentration from 2 days to 7 days following SC pump implantation (Cₛₛ). The following studies were completed, rhFSH 01-rhFSH 03.

The single dose pharmacokinetics of rhFSH in rats following a single IV dose of 0.5 μg/0.5 mL (actual dose: 0.455 μg/0.5 mL) were determined: mean AUC₀₋ₗₐₛₜ and AUC_{0-infinity} were 3535 and 3852 mIU/hr/mL, respectively, and alpha and beta half-lives for the distribution and elimination phases were 0.46±0.19 and 4.02±1.04 hr, respectively, with a clearance of 36.43 mL/hr/kg Three doses of rhFSH were given as a SC single bolus. The rhFSH was absorbed in a dose proportional manner with maximum mean serum concentration at approximately 9.4 to 10 hours. A comparison of the mean PK parameters from the three dose groups A, B, and C of the SC bolus treatment show alpha and beta half-lives of 5.5±0.97 and 8.0±1.6 hr for the 10 μg dose group (actual dose: 9:02 μg); and 4.8+1.6 and 8.6±1.3 hr for the 5 μg dose group (actual dose: 4.55 μg); and 5.4±0.98 and 10.3+5.0 hr for the 1 μg dose group (actual dose: 1 μg) the Tₘₐₓ values were 9.5±0.4, 9.4±0.6 and 10.0±0.9 hr per group, respectively. The mean Cₘₐₓ values 235127 mIU/mL for the 10 μg group, 90±9.3 mIU/mL for the 5 μg group, and 16.1±2.0, for the 1 μg group. The mean bioavailibilities of the SC bolus groups were 82.2±6.7, and 63.7±18.7 percent, respectively. Cₘₐₓ, AUC, and relative bioavailability all increased with increasing dose in a slightly non-proportional manner.

In an osmotic pump study, the pharmacokinetics of rhFSH in non-immunosuppressed rat model versus an immunosuppressed rat model were first compared. Two groups were compared each received 0.25 μg of rhFSH infused per hour (actual dose: 0.273 μg/hr) with one group receiving Sandimmune Cyclosporine intra-peritoneal (IP). No significant differences between groups B and D were noticed.

In addition, sustained release levels from the osmotic pump were assess for all doses and the 0.25 μg/hr immunosuppressed group. A mean Cₛₛ (from day 2 to day 7) of 149±5.2 mIU/mL for the 0.5 μg/hr group, 70±4.1 mIU/mL for the 0.25 μg/hr group, 13±1.8 mIU/mL for the 0.05 μg/hr group, and 79±7.1 mIU/mL for the 0.25 μg/hr in the immunosuppressed group. The absolute bioavailability for the four groups ranged from 37% to 42%; and relative bioavailability based upon the SC bolus doses ranged from 47% to 57%. Linearity was observed for group CSS values throughout the study.

### EXAMPLE 7

### Pharmacokinetic Profiles in Rats of Lots 2-14, 1-11, and 5-5, and Comparison with Human Data

TABLE X

| | | Mol. | | | |
|---|---|---|---|---|---|
| Formulation | PLG | Wt. | Sugar | salt | % FSH |
| 1-11 | 2M | 18 | sucrose | sodium | 0.51 |
| | (methyl ester) | | | phosphate | |
| 2-14 | 2A | 10 | sucrose | sodium | 0.54 |
| | (acid) | | | phosphate | |
| 5-5 | 2A:2M | 16 | sucrose | sodium | 0.55 |
| | (1:3 blend) | | | phosphate | |

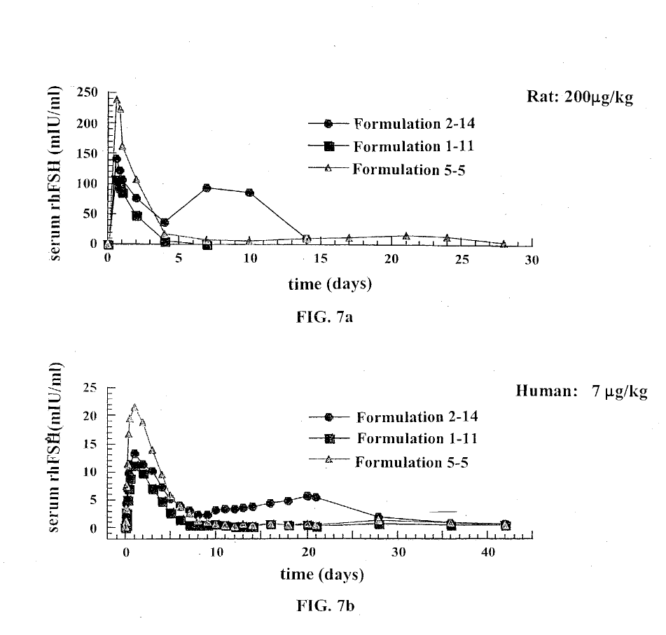

Each formulation from Table X was injected into 3 male Sprague-Dawley rats at a dose of 200 μg/kg of sustained release composition. Serum samples were collected periodically and the rhFSH serum concentration was determined using the MAIAclone IRMA assay. FIG. 7a shows the serum concentration of rhFSH versus time in days following administration of the indicated formulations. FIG. 7b shows the human data of serum concentration of rhFSH versus time in days following subcutaneous administration of the same formulations at a dose of 7 μg/kg.

All references cited herein are incorporated by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A composition for the sustained release of FSH comprising:
a) a poly(lactide-co-glycolide) copolymer having a molecular weight from about 5 kD to about 40 kD; and
b) a stabilized FSH formulation comprising FSH and at least one sugar;
wherein the stabilized FSH formulation is dispersed within the polymer.

2. The composition of claim 1, wherein the FSH is present from about 0.05% (w/w) to about 15% (w/w) of the total dry weight of the sustained release composition.

3. The composition of claim 1 wherein the FSH is present in the stabilized formulation from about 1% (w/w) to about 30% (w/w) of the total dry weight of the stabilized formulation.

4. The composition of claim 3, wherein the FSH is present in the stabilized formulation from about 3% (w/w) to about 30% (w/w) of the total dry weight of the stabilized formulation.

5. The composition of claim 1, wherein the composition is in the form of microparticles.

6. The composition of claim 1, wherein the sugar is present from about 50% (w/w) to about 99% (w/w) of the total dry weight of the stabilized formulation.

7. The composition of claim 6, wherein the sugar is present from about 70% (w/w) to about 97% (w/w) of the total dry weight of the stabilized formulation.

8. The composition of claim 1 wherein the sugar is a disaccharide.

9. The composition of claim 8, wherein the disaccharide is sucrose, lactose or trehalose.

10. The composition of claim 1 wherein the stabilized FSH formulation further comprises at least one buffer salt.

11. The composition of claim 10, wherein the buffer salt is present in the stabilized formulation from about 1 (w/w) to about 10% (w/w) of the total dry weight of the formulation.

12. The composition of claim 10, wherein the buffer salt is a phosphate buffer salt.

13. The composition of claim 1 wherein the FSH is released for at least 5 days.

14. The composition of claim 1 wherein the FSH is released for at least 30 days.

15. The composition of claim 1, wherein the poly(lactide-co-glycolide) copolymer has a molecular weight from about 100 kD to about 20 kD.

16. The composition of claim 15, wherein the poly(lactide-co-glycolide) copolymer has an acid terminal group.

17. The composition of claim 15, wherein the poly(lactide-co-glycolide) copolymer has a methyl ester terminal group.

18. The composition of claim 1, wherein the poly(lactide-co-glycolide) copolymer is a blend comprising at least one acid terminal end group poly(lactide-co-glycolide) and at least one methyl ester terminal poly(lactide-co-glycolide).

19. The composition of claim 18 wherein the blend of copolymers is a ratio of 1 acid terminal end group to 3 ester terminal end groups.

20. The composition of claim 1, wherein the stabilized FSH formulation comprises about 1% (w/w) to about 30% (w/w) FSH, about 50% to about 99% sugar and about 1% to about 10% buffer salt.

21. A method for delivery of FSH to a patient in need of such delivery comprising administering to said patient a therapeutically effective amount of a composition for the sustained release of FSH, comprising
a) a poly(lactide-co-glycolide) copolymer having a molecular weight from about 5 kD to about 40 kD; and
b) a stabilized FSH formulation comprising FSH and at least one sugar;
wherein the stabilized FSH formulation is dispersed within the polymer.

22. The method of claim 21, wherein the FSH is present from about 0.05% (w/w) to about 15% (w/w) of the total dry weight of the sustained release composition.

23. The method of claim 21 wherein the FSH is present in the stabilized formulation from about 1% (w/w) to about 30% (w/w) of the total dry weight of the stabilized formulation.

24. The method of claim 23, wherein the FSH is present in the stabilized formulation from about 3% (w/w) to about 30% (w/w) of the total dry weight of the stabilized formulation.

25. The method of claim 21, wherein the composition is in the form of microparticles.

26. The method of claim 21, wherein the sugar is present from about 50% (w/w) to about 99% (w/w) of the total dry weight of the stabilized formulation.

27. The method of claim 26, wherein the sugar is present from about 70% (w/w) to about 97% (w/w) of the total dry weight of the stabilized formulation.

28. The method of claim 21 wherein the sugar is a disaccharide.

29. The method of claim 28, wherein the disaccharide is sucrose, lactose or trehalose.

30. The method of claim 21 wherein the stabilized FSH formulation further comprises at least one buffer salt.

31. The method of claim 30, wherein the buffer salt is present in the stabilized formulation from about 1 (w/w) to about 10% (w/w) of the total dry weight of the formulation.

32. The method of claim 30, wherein the buffer salt is a phosphate buffer salt.

33. The method of claim 21 wherein the FSH is released for at least 5 days.

34. The method of claim 21, wherein the FSH is released for at least 30 days.

35. The method of claim 21, wherein the poly(lactide-co-glycolide) copolymer has a molecular weight from about 110 kD to about 20 kD.

36. The method of claim 35, wherein the poly(lactide-co-glycolide) copolymer has an acid terminal group.

37. The method of claim 35, wherein the poly(lactide-co-glycolide) copolymer has a methyl ester terminal group.

38. The method of claim 21, wherein the stabilized FSH formulation comprises about 1% (w/w) to about 30% (w/w) FSH, about 50% to about 99% sugar and about 1% to about 10% buffer salt.

39. The method of claim 21, wherein the poly(lactide-co-glycolide) copolymer is a blend comprising at least one acid terminal end group poly(lactide-co-glycolide) and at least one methyl ester terminal poly(lactide-co-glycolide).

40. The method of claim 39 wherein the blend of copolymers is a ratio of 1 acid terminal end group to 3 ester terminal end groups.

41. A method for providing a therapeutically effective blood level of FSH in a patient for a sustained period, comprising:
a) a poly(lactide-co-glycolide) copolymer having a molecular weight from about 5 kD to about 40 kD; and
b) a stabilized FSH formulation comprising FSH and at least one sugar;
wherein the stabilized FSH formulation is dispersed within the polymer.

42. The method of claim 41, wherein the FSH is present from about 0.05% (w/w) to about 15% (w/w) of the total dry weight of the sustained release composition.

43. The method of claim 41 wherein the FSH is present in the stabilized formulation from about 1% (w/w) to about 30% (w/w) of the total dry weight of the stabilized formulation.

44. The method of claim 43, wherein the FSH is present in the stabilized formulation from about 3% (w/w) to about 30% (w/w) of the total dry weight of the stabilized formulation.

45. The method of claim 41, wherein the composition is in the form of microparticles.

46. The method of claim 41, wherein the sugar is present from about 50% (w/w) to about 99% (w/w) of the total dry weight of the stabilized formulation.

47. The method of claim 46, wherein the sugar is present from about 70% (w/w) to about 97% (w/w) of the total dry weight of the stabilized formulation.

48. The method of claim 41 wherein the sugar is a disaccharide.

49. The method of claim 48, wherein the disaccharide is sucrose, lactose or trehalose.

50. The method of claim 41 wherein the stabilized FSH formulation further comprises at least one buffer salt.

51. The method of claim 50, wherein the buffer salt is present in the stabilized formulation from about 1 (w/w) to about 10% (w/w) of the total dry weight of the formulation.

52. The method of claim 50, wherein the buffer salt is a phosphate buffer salt.

53. The method of claim 41 wherein the FSH is released for at least 5 days.

54. The method of claim 41, wherein the FSH is released for at least 30 days.

55. The method of claim 41, wherein the poly(lactide-co-glycolide) copolymer has a molecular weight from about 10 kD to about 20 kD.

56. The method of claim 55, wherein the poly(lactide-co-glycolide) copolymer has an acid terminal group.

57. The method of claim 55, wherein the poly(lactide-co-glycolide) copolymer has a methyl ester terminal group.

58. The method of claim 41, wherein the stabilized FSH formulation comprises about 1% (w/w) to about 30% (w/w) FSH, about 50% to about 99% sugar and about 1% to about 10% buffer salt.

59. The method of claim 41, wherein the poly(lactide-co-glycolide) copolymer is a blend comprising at least one acid terminal end group poly(lactide-co-glycolide) and at least one methyl ester terminal poly(lactide-co-glycolide).

60. The method of claim 59 wherein the blend of copolymers is a ratio of 1 acid terminal end group to 3 ester terminal end groups.

61. A method of promoting the maturation of follicles in the ovary of a patient, comprising administering a therapeutically effective amount to a patient in need of treatment, a therapeutically effective amount of a sustained release composition comprising:
a) a poly(lactide-co-glycolide) copolymer having a molecular weight from about 5 kD to about 40 kD; and
b) a stabilized FSH formulation comprising FSH and at least one sugar;
wherein the stabilized FSH formulation is dispersed within the polymer.

62. The method of claim 61, wherein the FSH is present from about 0.05% (w/w) to about 15% (w/w) of the total dry weight of the sustained release composition.

63. The method of claim 61 wherein the FSH is present in the stabilized formulation from about 1% (w/w) to about 30% (w/w) of the total dry weight of the stabilized formulation.

64. The method of claim 63, wherein the FSH is present in the stabilized formulation from about 3% (w/w) to about 30% (w/w) of the total dry weight of the stabilized formulation.

65. The method of claim 61, wherein the composition is in the form of microparticles.

66. The method of claim 61, wherein the sugar is present from about 50% (w/w) to about 99% (w/w) of the total dry weight of the stabilized formulation.

67. The method of claim 66, wherein the sugar is present from about 70% (w/w) to about 97% (w/w) of the total dry weight of the stabilized formulation.

68. The method of claim 61 wherein the sugar is a disaccharide.

69. The method of claim 68, wherein the disaccharide is sucrose, lactose or trehalose.

70. The method of claim 61 wherein the stabilized FSH formulation further comprises at least one buffer salt.

71. The method of claim 70, wherein the buffer salt is present in the stabilized formulation from about 1 (w/w) to about 10% (w/w) of the total dry weight of the formulation.

72. The method of claim 70, wherein the buffer salt is a phosphate buffer salt.

73. The method of claim 61 wherein the FSH is released for at least 5 days.

74. The method of claim 61, wherein the FSH is released for at least 30 days.

75. The method of claim 61, wherein the poly(lactide-co-glycolide) copolymer has a molecular weight from about 10 kD to about 20 kD.

76. The method of claim 75, wherein the poly(lactide-co-glycolide) copolymer has an acid terminal group.

77. The method of claim 75, wherein the poly(lactide-co-glycolide) copolymer has a methyl ester terminal group.

78. The method of claim 61, wherein the stabilized FSH formulation comprises about 1% (w/w) to about 30% (w/w) FSH, about 50% to about 99% sugar and about 1% to about 10% buffer salt.

79. The method of claim 61, wherein the poly(lactide-co-glycolide) copolymer is a blend comprising at least one acid terminal end group poly(lactide-co-glycolide) and at least one methyl ester terminal poly(lactide-co-glycolide).

80. The method of claim 79 wherein the blend of copolymers is a ratio of 1 acid terminal end group to 3 ester terminal end groups.

81. A method of promoting spermatogenesis in the testes of a patient, comprising administering a therapeutically effective amount to a patient in need of treatment, a therapeutically effective amount of a sustained release composition comprising:
a) a poly(lactide-co-glycolide) copolymer having a molecular weight from about 5 kD to about 40 kD; and
b) a stabilized FSH formulation comprising FSH and at least one sugar;
wherein the stabilized FSH formulation is dispersed within the polymer.

82. The method of claim 81, wherein the FSH is present from about 0.05% (w/w) to about 15% (w/w) of the total dry weight of the sustained release composition.

83. The method of claim 81 wherein the FSH is present in the stabilized formulation from about 1% (w/w) to about 30% (w/w) of the total dry weight of the stabilized formulation.

84. The method of claim 83, wherein the FSH is present in the stabilized formulation from about 3% (w/w) to about 30% (w/w) of the total dry weight of the stabilized formulation.

85. The method of claim 81, wherein the composition is in the form of microparticles.

86. The method of claim 81, wherein the sugar is present from about 50% (w/w) to about 99% (w/w) of the total dry weight of the stabilized formulation.

87. The method of claim 86, wherein the sugar is present from about 70% (w/w) to about 97% (w/w) of the total dry weight of the stabilized formulation.

88. The method of claim 81 wherein the sugar is a disaccharide.

89. The method of claim 88, wherein the disaccharide is sucrose, lactose or trehalose.

90. The method of claim 81 wherein the stabilized FSH formulation further comprises at least one buffer salt.

91. The method of claim 90, wherein the buffer salt is present in the stabilized formulation from about 1 (w/w) to about 10% (w/w) of the total dry weight of the formulation.

92. The method of claim 90, wherein the buffer salt is a phosphate buffer salt.

93. The method of claim 81 wherein the FSH is released for at least 5 days.

94. The method of claim 81, wherein the FSH is released for at least 30 days.

95. The method of claim 81, wherein the poly(lactide-co-glycolide) copolymer has a molecular weight from about 10 kD to about 20 kD.

96. The method of claim 95, wherein the poly(lactide-co-glycolide) copolymer has an acid terminal group.

97. The method of claim 95, wherein the poly(lactide-co-glycolide) copolymer has a methyl ester terminal group.

98. The method of claim 81, wherein the stabilized FSH formulation comprises about 1% (w/w) to about 30% (w/w) FSH, about 50% to about 99% sugar and about 1% to about 10% buffer salt.

99. The method of claim 81, wherein the poly(lactide-co-glycolide) copolymer is a blend comprising at least one acid terminal end group poly(lactide-co-glycolide) and at least one methyl ester terminal poly(lactide-co-glycolide).

100. The method of claim 99 wherein the blend of copolymers is a ratio of 1 acid terminal end group to 3 ester terminal end groups.

101. A method of treating fertility disorders, comprising administering to a patient in need treatment a therapeutically effective amount of a sustained release composition comprising:
a) a poly(lactide-co-glycolide) copolymer having a molecular weight from about 5 kD to about 40 kD; and
b) stabilized FSH formulation comprising FSH and at least one sugar wherein the FSH is dispersed therein.

102. The method of claim 101, wherein the FSH is present from about 0.05% (w/w) to about 15% (w/w) of the total dry weight of the sustained release composition.

103. The method of claim 101 wherein the FSH is present in the stabilized formulation from about 1% (w/w) to about 30% (w/w) of the total dry weight of the stabilized formulation.

104. The method of claim 103, wherein the FSH is present in the stabilized formulation from about 3% (w/w) to about 30% (w/w) of the total dry weight of the stabilized formulation.

105. The method of claim 101, wherein the composition is in the form of microparticles.

106. The method of claim 101, wherein the sugar is present from about 50% (w/w) to about 99% (w/w) of the total dry weight of the stabilized formulation.

107. The method of claim 106, wherein the sugar is present from about 70% (w/w) to about 97% (w/w) of the total dry weight of the stabilized formulation.

108. The method of claim 101 wherein the sugar is a disaccharide.

109. The method of claim 108, wherein the disaccharide is sucrose, lactose or trehalose.

110. The method of claim 101 wherein the stabilized FSH formulation further comprises at least one buffer salt.

111. The method of claim 110, wherein the buffer salt is present in the stabilized formulation from about 1 (w/w) to about 10% (w/w) of the total dry weight of the formulation.

112. The method of claim 110, wherein the buffer salt is a phosphate buffer salt.

113. The method of claim 101 wherein the FSH is released for at least 5 days.

114. The method of claim 101, wherein the FSH is released for at least 30 days.

115. The method of claim 101, wherein the poly(lactide-co-glycolide) copolymer has a molecular weight from about 10 kD to about 20 kD.

116. The method of claim 115, wherein the poly(lactide-co-glycolide) copolymer has an acid terminal group.

117. The method of claim 115, wherein the poly(lactide-co-glycolide) copolymer has a methyl ester terminal group.

118. The method of claim 101, wherein the stabilized FSH formulation comprises about 1% (w/w) to about 30% (w/w) FSH, about 50% to about 99% sugar and about 1% to about 10% buffer salt.

119. The method of claim 101, wherein the poly(lactide-co-glycolide) copolymer is a blend comprising at least one acid terminal end group poly(lactide-co-glycolide) and at least one methyl ester terminal poly(lactide-co-glycolide).

120. The method of claim 119 wherein the blend of copolymers is a ratio of 1 acid terminal end group to 3 ester terminal end groups.

121. A method for forming a composition for the sustained release of FSH comprising:
a) dissolving a poly(lactide-co-glycolide) copolymer having a molecular weight from about 5 kD to about 40 kD in a polymer solvent to form a polymer solution;
b) adding a stabilized FSH formulation comprising FSH and at least one sugar to the polymer solution to form a polymer/stabilized FSH formulation mixture, wherein the FSH is present at a final concentration of between about 0.05% (w/w) and about 15% (w/w) of the dry weight of the composition;
c) removing the solvent from the polymer/stabilized FSH mixture; and
d) solidifying the polymer to form a polymer matrix containing the stabilized FSH formulation dispersed therein.

122. The method of claim 121 further comprising the steps of:
a) forming droplets of the polymer/stabilized FSH formulation mixture;
b) freezing the droplets of the polymer/stabilized FSH formulation mixture wherein said forming and freezing steps are performed prior to removal of the solvent.

123. The method of claim 121 wherein the solvent is removed by extraction with an extraction solvent.

124. The method of claim 121 wherein the droplets are microdroplets.

125. The method of claim 121 wherein the extraction solvent is ethanol.

126. The method of claim 121 wherein the sustained release composition is in the form of microparticles.

127. A composition for the sustained release of FSH prepared by a method comprising:
a) dissolving a poly(lactide-co-glycolide) copo;ymer having a molecular weight from about 5 kD to about 40 kD in a polymer solvent to form a polymer solution;
b) adding a stabilized FSH formulation to achieve a mixture comprising a polymer/stabilized FSH formulation; and
c) removing the solvent from the polymer/stabilized FSH mixture, thereby forming a polymer matrix containing solid FSH dispersed therein.

128. The composition of claim 127 wherein the method further comprising the steps of:
a) forming droplets of the polymer/stabilized FSH formulation mixture;
b) freezing the droplets of the polymer/stabilized FSH formulation mixture wherein said forming and freezing steps are preformed prior to removal of the solvent.

129. The composition of claim 127 wherein the solvent is removed by extraction with an extraction solvent.

130. The composition of claim 129 wherein the droplets are microdroplets.

131. The composition of claim 127 wherein the extraction solvent is ethanol.

132. The composition of claim 127 which is in the form of microparticles.
